(19) United States
(12) Patent Application Publication
(10) Pub. No.: US 2011/0118555 A1
(43) Pub. Date: May 19, 2011

(54) SYSTEM AND METHODS FOR SCREENING, TREATING, AND MONITORING PSYCHOLOGICAL CONDITIONS

(76) Inventors: Abhijit Dhumne, Oakton, VA (US); Hans Kristofer Breville, Arlington, VA (US)

(21) Appl. No.: 12/770,375

(22) Filed: Apr. 29, 2010

Related U.S. Application Data
(60) Provisional application No. 61/173,787, filed on Apr. 29, 2009.

(51) Int. Cl. A61B 5/00

(52) U.S. Cl. 600/300

(57) ABSTRACT

A system for and method of remote monitoring, screening, assessment and treatment of patients having a mental health illness such as post-traumatic stress disorders or other traumatic stress injury and co-occurring symptomatology. Patients in constant communication with one or more healthcare professionals through a wireless network, complete executable programs on their patient handheld electronic devices and transmit the results of the executable programs to their supervising mental health professional. The mental health professionals review and analyze the collected patient data to make clinical assessments of the patients' mental health status.

### CROSS REFERENCE TO RELATED U.S. PATENT APPLICATIONS

This application claims the benefit of priority from provisional application No. 61/173,787 filed Apr. 29, 2009, which is incorporated herein by reference.

### BACKGROUND OF INVENTION

1. Field of the Invention

The present invention relates to an electronic system for monitoring, assessing, screening and treating mental illness in patients. More particularly, the present invention relates to a clinical system for and method of monitoring assessing, screening and treating post-traumatic stress disorder, co-occurring traumatic stress injuries, and symptoms thereof

2. Description of the Prior Art

A mental disorder, also commonly referred to as a mental illness, is a psychological or behavioral pattern that occurs in a person and is thought to cause distress or disability that is not expected as part of normal development or culture. Mental disorders are common in the United States and internationally. According to the National Institutes of Mental Health (NIMH), it is estimated that 26.2 percent of Americans ages 18 and older—about one in four adults—experience a diagnosable mental disorder in a given year. This figure translates to approximately 58 million people in the United States. In fact, it is estimated that mental disorders are the leading cause of disability in the United States and Canada for individuals from 15-44 years old.

The Diagnostic and Statistical Manual of Mental Disorders (DSM) organizes mental disorders by category based on similar features, etiology, and/or functional impairments. Some of the categories include, for example, dissociative disorders, mood disorders, psychotic disorders, eating disorders, developmental disorders, personality disorders, and anxiety disorders. Anxiety disorders, in particular, include panic disorder, obsessive-compulsive disorder, post-traumatic stress disorder, generalized anxiety disorder, and phobias, such as social phobia, agoraphobia, and other specific phobias. For example, Attention-Deficit Hyperactivity Disorder (ADHD) is listed in the DSM under disorders in childhood or infancy (DSM 314.9), while Post-Traumatic Stress Disorder (PTSD) is found under anxiety disorders (DSM 309.81).

More specifically, PTSD, which has been recognized in the past as railway spine, stress syndrome, shell shock, battle fatigue, traumatic war neurosis, or post-traumatic stress s syndrome (PTSS), is different from most mental-health diagnoses because it is tied to a particular specified life experience identified as a trauma or traumatic event. It is believed to develop after exposure to one or more traumatic events whereby the individual is, or perceives that they were, threatened or caused grave physical harm, or more generally, that involved the potential for death or serious injury resulting in intense fear, helplessness, or honor. Often, it is a severe and ongoing cognitive and emotional reaction to an extreme psychological or emotional trauma. This stressor may involve someone's actual death, a threat to the patient's or someone else's life, serious physical injury, an unwanted sexual act, or a threat to physical or psychological integrity, overwhelming psychological defenses.

PTSD can be relatively highly prevalent in military personnel, and the rates of incidence and diagnosis are increasing. One study indicates that while the prevalence of PTSD among the general U.S. population is about 5 percent, the prevalence in Vietnam veterans ranges as high as 20 to 30 percent. According to data published by the Pentagon, the total number of United States military personnel serving in Iraq and Afghanistan who were diagnosed with PTSD increased by 50 percent from 2006 to 2007 to nearly 40,000. These numbers translate to approximately 30 percent of all U.S. troops serving in Iraq and Afghanistan suffer from PTSD. It should be noted, however, that the total reported numbers may be a fraction of total PTSD cases due to underreporting and misdiagnosis.

A diagnosis of PTSD includes specified symptoms within four symptom cluster categories: re-experiencing, avoidance, numbing, and arousal. Commonly, diagnostic symptoms include re-experiencing the traumatic event in various forms, such as flashbacks and intrusive unwanted memories, avoidance of stimuli associated with the trauma (such as through avoidance of people, conversations, or situations, or through emotional numbing symptoms), increased arousal such as difficulty falling or staying asleep often associated with nightmares, anger and hyper-vigilance. The symptoms last more than one month and are often chronic in nature and cause significant impairment in social, occupational, or other important areas of functioning (e.g., problems with work and relationships). Conventionally, PTSD patients undergo one or more in-person interventions, including a psycho-education of the mental disorder, exposure therapies including cognitive processing therapy and prolonged exposure, cognitive behavioral therapy, motivational interviewing, behavioral activation, anxiety and symptom management skills, supportive therapy, behavioral couple's therapy, and integrated substance abuse treatment, among others. PTSD has been found to have high co-morbidity with other disorders including mood and discrete anxiety disorders and substance abuse disorders. While manualized short term therapies exist, treatment is frequently long term and complex, often entailing both inpatient and outpatient treatment modalities.

The closure of hundreds of acute care beds for mental health patients, and for military populations, the tremendous pressures on the Veterans Affairs healthcare system have highlighted a lack of continuity, communication and general cohesion of services for patients suffering from mental illness. Patients with mental health problems, such as PTSD, are typically seen by mental health professionals (also commonly and interchangeably referred to as “mental health workers,” “healthcare workers (HCW),” “psychologists,” “psychiatrists,” “clinicians,” “physicians,” “doctors,” “practitioners,” “therapists,” “counselors,” or the like) on a periodic basis rather than an everyday basis. Thus, mental health professionals only have an opportunity to monitor their patients on days in which patients come to the office. Patients typically receive no specific care on other days. Patients suffering from mental illness may display little or no symptoms of their clinical phenomena during a scheduled, in-person evaluation, thus leaving the possibility of highly variable behavior on a day-to-day basis. As a result, the only time the mental health professional would be aware of dangerous behavior would likely be after the event, thereby effectively eliminating preventative treatment. Alternatively, patients who have felt the strain of a prolonged period without direct contact with the mental health provider may overreport symptoms during in-person sessions, leading to an increased burden on an already taxed system.

Mental health professionals do not currently have a way to ensure oversight of their patients between evaluations or over the long-term. The result is that there is little or no monitoring of their mental status and that many patients, particularly in the population experiencing PTSD or PTSD-like symptoms, are poorly managed and under-treated, and in some cases, are left untreated. Patient compliance and fidelity with treatment between clinical treatment sessions is a source of significant concern. There is a lack of systems and methods for following up patients who are non-compliant with their therapy, medication, or treatment regimen as a whole. It is widely believed that poor compliance will lead to treatment failure and the return of or worsening of clinical symptoms.

In view of the foregoing, it is clear that there is a need for a remote PTSD monitoring and treatment system, which can assist patients in need of therapy and/or intervention on a more consistent and ongoing basis. More specifically, there is a need in the art for a technology that ensures that the patient and doctor can have an expanded link between one another, thus allowing the physician to monitor or track an individual's progress over a period of time. There is a need in the art to provide such health services in a reliable, cost efficient manner while providing it to as many patients in need as possible. Although there may be various types of prior remote, electronic health tracking systems available in the marketplace, none is directed to acute stress disorders, PTSD or to adult, military-related populations.

### SUMMARY OF THE INVENTION

The present invention advantageously provides systems and methods to provide remote healthcare to patients suffering from a mental illness, such as anxiety orders, particularly acute stress disorders or PTSD. Accordingly, the present invention is directed to providing a system for and method of monitoring, assessing, screening and treating PTSD or symptoms thereof, which overcome the problems associated with the prior art. One aspect of the present invention is to provide a computer-implemented system for monitoring post-traumatic stress disorder in a patient in need. The system includes at least one electronic, portable patient device, having at least an input mechanism to enter patient information, a display to review the patient information and communication apparatus, in communication with a computer-implemented processor accessible by one or more mental health professionals and located remote from the patient device. The computer-implemented processor is configured to transmit one or more executable programs of a patient clinical module to the patient device to receive executed programs from the patient. Additionally, the patient clinical modules provide therapies for mental health care for PTSD or symptoms thereof, selected from the group including a therapy module to monitor and detect a post-traumatic stress disorder symptoms and an informational module to deliver educational material about post-traumatic stress disorder, for example.

In accordance with one embodiment of the present invention, a method for treating post-traumatic stress disorder in a patient in need, comprising the steps of providing a patient device to the patient to collect patient information regarding PTSD and providing a PTSD screening functionality on a preferably handheld device, wherein the screening functionality is configured with threshold clinical and diagnostic values to determine a degree of post-traumatic disorder symptoms or to determine patient progress using relative values. Additional steps include receiving collected patient information through a communication network on at least one computer-implemented processor accessible by a mental health professional and processing the patient information to determine the clinical severity and magnitude of symptoms associated with PTSD and co-occurring traumatic stress injuries. In some instances, the steps include alerting a first responder if one or more predetermined threshold clinical values are exceeded by the patient.

In accordance with another embodiment of the present invention, a method of remotely monitoring PTSD in a patient includes the steps of providing an entity associated with at least one mental health professional with a system to remotely diagnose and monitor PTSD in at least one patient through one or more computer-executable programs directed to PTSD, the programs prompting interaction by the patient through a communication network connecting a patient device with a central monitoring computer, wherein the patient device and central monitoring computer transmit and receive patient mental health assessment data providing information on the patient's mental health. The method also includes the steps of collecting the information at the central monitoring computer as a component of a patient profile for treatment of PTSD; assessing that information to determine efficacy and scope of the treatment; and customizing the treatment by updating one or more computer-executable programs to be addressed by the patient as part of therapy for PTSD.

The present invention will now be described in greater detail, with frequent reference being made to the drawings identified below in which identical numerals represent identical elements.

### BRIEF DESCRIPTION OF THE DRAWINGS

Further advantageous features of the present invention will become more apparent with the following detailed description when taken with reference to the accompanying drawings in which:

FIG. 1 is a functional block diagram of hardware, software, or a combination thereof that may be implemented in one or more computer systems or other processing systems to carry out the functionality for remote monitoring and related features, in accordance with one embodiment of the present invention;

FIG. 2 is a functional block diagram of an exemplary system of various hardware o components and other features, in accordance with an embodiment of the present invention;

FIG. 3 is an exemplary network schematic, in accordance with one embodiment of the present invention;

FIG. 4a illustrates an exemplary schematic of terminal, in accordance with one embodiment of the present invention;

FIG. 4b illustrates exemplary patient modules, in accordance with one embodiment of the present invention;

FIG. 4c provides an exemplary illustration of the patient device and graphical user interface thereof, in accordance with one embodiment of the present invention;

FIG. 4d provides an exemplary PTSD Checklist (PCL), in accordance with one embodiment of the invention;

FIG. 4e provides an exemplary Mississippi Scale, in accordance with one embodiment of the invention;

FIG. 5a provides exemplary steps involved in the method of diagnosing mental illness, in accordance with one embodiment of the present invention;

FIG. 5b provides exemplary steps involved in the method of operating the patient device, in accordance with one embodiment of the present invention;

FIG. 5c provides exemplary steps involved in the method of monitoring a patient's mental health, in accordance with one embodiment of the present invention;

FIG. 5d provides additional exemplary steps involved in the method of monitoring a patient's mental health, in accordance with one embodiment of the present invention;

FIG. 5e provides exemplary steps involved in the method of completing the monitoring a patient's mental health, in accordance with one embodiment of the present invention; and

FIGS. 6a-f provide exemplary flowcharts illustrating diagnosis and treatment of PTSD in military personnel, in accordance with an embodiment of the present invention.

### DETAILED DESCRIPTION OF THE PREFERRED EMBODIMENT(S)

The following description is presented to enable any person of ordinary skill in the art to practice the present invention. Modifications to the preferred embodiment will be readily apparent to those of ordinary skill in the art, and the disclosure set forth herein may be applied to other embodiments and applications without departing from the spirit and scope of the present invention and appended claims. Thus, the present invention is not intended to be limited to the embodiments described, but is to be accorded the broadest scope consistent with the disclosure set forth herein.

As discussed above, the present invention relates to a system and method for remotely monitoring, treating, and preferably building skills for living and coping with a mental illness, more preferably, acute stress disorder, and most preferably PTSD. Aspects of the present invention provide a novel system and method of telemedicine (also commonly referred to as “telehealth”) using electronic communications and information technology to provide and support mental healthcare, particularly to populations separated from healthcare personnel or facilities due to logistical issues, such as geographical distance or time burden. Accordingly, the system and methods of the present invention facilitate remote communication between mental health professionals and their patients, as an alternative, or an adjunctive treatment model, to traditional in-person counseling and outreach using the telephone. The present invention provides valid, reproducible, and reliable measures and patient mental health assessments. Additionally, the present invention improves the standardization and quantification of symptom reporting.

Embodiments of the present invention implement a hardware and software platform establishing a communication link between patients and clinicians. The link will provide a monitoring functionality, for example, to provide screen and identify presence of symptoms or symptom clusters; to facilitate transmission of reliable, empirical evidence; to increase access to remote patients; to supplement and/or replace medical care, typically provided through in-person meetings, to track and collect longitudinal data; to provide regular or continuous treatment; and to alert first responders to emergency situations. Moreover, embodiments may allow or facilitate a comparison of therapeutic approaches, gauge the degree of commitment to treatment; assess usage in view of patient profiles (demographic, geographic, etc.); increase early identification and intervention; enhance the standardization and quantification of symptom reporting; and assess the relationship between deployment and symptom intensity or illness severity.

In one embodiment of the present invention, a plurality of patients is provided with a wireless hardware and software platform to establish a communication link between the patient's doctor, psychiatrist, psychologist, or regular mental health professional, preferably through a central monitoring location, such as electronic terminal (or a network of electronic terminals) used by the mental health professional personally or at the office. The system and methods provide a screening and diagnosis functionality to identify the reported presence of symptom clusters, provide a platform for treatment and to practice skills learned in treatment, and a monitoring and tracking functionality to observe patients and mental illness symptoms continually and longitudinally, alerting interested parties, such as mental health professionals and/or first responders when a patient's reported symptoms are significantly elevated, or otherwise abnormal.

Preferred embodiments of the present invention provide one or more of the following functionalities: screening, assessment, diagnosis, monitoring, tracking, management, treatment, prophylaxis, prevention, skills building (i.e., skill practice), training and risk management. For the purposes of this invention, these functionalities are generally associated with their common usage of the term, as used in the medical and healthcare fields. The definitions below provide further usage of the terms describing the functionalities which only supplement their common meaning and should not be construed to limit their meaning in any way.

As used herein, the term “screening” generally encompasses one or more strategies used in a population to identify symptoms generally associated with a disease in individuals without signs or symptoms of that disease. In some instances, screening may be performed on those without any clinical indication of disease. The term “assessment” or “assessing” generally relates to the screening or diagnosis process as it encompasses an evaluation of the health status of an individual by performing a physical, psychological, or other clinical examination after obtaining a health history. Various laboratory diagnostic and functional tests may also be ordered to confirm a clinical impression or to screen for dysfunction.

As used herein, the term “monitoring” broadly encompasses strategies to detect, observe, and record a patient's vital signs, symptoms, health or other disease-related measurements. Data collected may be stored in an electronic database and subject to statistical and clinical review. The term “tracking” is one aspect of monitoring which connotes continuous, or at least regular, monitoring of the same patient over time, such as at least in two instances, preferably to determine patterns or to ascertain longitudinal patient data.

As used herein, the term “treatment” or “treating” encompasses any treatment of a disease or disorder in a human, including: preventing or protecting against the disease or disorder of further development thereof, that is, causing the clinical symptoms not to develop; inhibiting the disease or disorder; arresting or suppressing the development of clinical symptoms; and/or relieving the disease or disorder; and causing the regression of clinical symptoms. In some embodiments, the term “treatment” or “treating” includes ameliorating the symptoms of, curing or healing, and preventing the development of a given disease.

The term “prophylaxis” is intended as an element of “treatment” to encompass both “preventing” and “suppressing,” as defined herein. It will be understood by those skilled in the art that in human medicine it is not always possible to distinguish between “preventing” and “suppressing” since the ultimate inductive event or events may be unknown, latent, or the patient is not ascertained until well after the occurrence of the event or events.

As used herein, the term risk management generally refers to a system for mitigating or reducing associated risk of mental health concerns, particularly PTSD in certain embodiments of this invention. These risks include, but are not limited to, psychological distress and self harm, up to and including suicide/suicidality or homicide/homicidality.

As used herein, the term “patient” (also commonly referred to in the art as “subject”) generally refers to a person in need of screening, diagnosis, monitoring, tracking, management, treatment, prophylaxis, prevention, skills building, and training related to one or more mental illnesses, such as PTSD. For the purposes of this invention, the patient is a human at any age. As provided for in certain embodiments of the present invention, the patient population covered by the present invention includes any patient susceptible to or suffering from a mental illness, particularly PTSD, with the mental and physical ability to maintain operation of a portable, preferably handheld, patient electronic device. In preferred embodiments, the present invention is directed toward current and former military personnel (e.g., soldiers), adult individuals with combat experience, and/or department of defense employees and contractors. In certain embodiments, the target population includes personnel with national security clearance. In the most preferred embodiments, the target population includes individuals attributing their PTSD to one or more traumatic circumstances related to their military service.

As used herein, the term “mental health professional” encompasses any party responsible for, or assisting with, the mental healthcare of a patient. Communications between a patient and mental health professional may be direct, or may include at least one intervening party affiliated with the mental health professional or acting on behalf of the mental health professional, such as, but not limited to, agents, employees, independent contractors, and other health professionals or facilities. In certain embodiments of the present invention, the patient device communicates with an electronic terminal for, or operated by, the mental health professional. In embodiments of the present invention, the mental health professional, his staff and/or affiliation, and any other party assisting the professional may be generically referred to herein as the “clinican,” “doctor's office” or “central office.” The central office is configured to receive and transmit communications to and from one or more patients, one or mental health professionals, and other affiliated parties. The central office may be a traditional office and may include one or more terminals (i.e., computers) that can receive patient communications through a wired or wireless network.

The system and methods described herein provide numerous benefits, such as: 1) facilitating continuous monitoring and tracking of one or more patients, particularly patients in at-risk populations; 2) allowing a limited number of health professionals to provide healthcare for a large population of patients; 3) implementing preventative medical measures to avoid or mitigate destructive behavior; 4) increasing access to healthcare by providing medical coverage to remote geographic areas; 5) effectively eliminating costly and disruptive travel by the healthcare professional or patient; 6) increasing patient compliance of mental health therapy and treatments; 7) promoting patient-record keeping; 8) reducing healthcare costs; 9) providing a means to alert healthcare workers or first response personnel in a prompt and efficient manner; 10) implementing a model of care known as distance or telehealth management whereby treatment is preferred or provided over distance by implementing or utilizing technological applications, thus increasing a patient's capacity to be treated even if he or she is distant from care and increasing a clinician's ability to reach patients in need who are distant or remote from services; and 11) providing mental health professionals with a tool to better serve their patients' many needs, thereby reducing burn out, such as through fatigue or vicarious traumatization and maintaining a pool of trained professionals to adequately serve the population of mental health patients. Embodiments of this invention allow a small community clinic to offer access to specialized interventions and specialists in PTSD, which the clinic would normally not be able to provide. Moreover, by allowing patients to undergo therapy at a location of their choice, embodiments of the prevention provide a way for housebound patients to get the help that they need.

A key benefit of the present invention is the ability to treat a large number of patients while reducing the costs and time consumption typically associated with one-on-one, in-person mental healthcare. Accordingly, embodiments of the present invention allow for the concurrent or substantially concurrent treatment of multiple patients by one mental health professional or one central office. In accordance with the present invention, the ratio of remotely monitored and treated patients to mental health professionals may range from about 1:1 to 150:1, preferably from 2:1 to 100:1 and more preferably from 4:1 to 50:1. It is also contemplated that more than one mental health professional may oversee one patient in certain embodiments. Additionally, it is further contemplated that multiple mental health working collectively, via a network, hospital, managed care or other setting, may provide care to a multitude of patients, from 100 to 1000 patients or more. Generally, in accordance with embodiments of the invention, the size of the patient population is only limited by the ability of a mental health professional to provide acceptable and valid mental healthcare and/or prescribed remote interaction.

Embodiments of the present invention include monitoring and/or treatment for PTSD for at least 1 week, preferably for at least 1 month, more preferably for at least 2 months, even more preferably for at least 3 months, and most preferably between 1 month and 24 months. In preferred embodiments, the duration of treatment of PTSD through remote healthcare corresponds to the same duration as traditional PTSD treatments. The duration of one type of PTSD therapy may be independent from the duration of other PTSD therapies. For example, two therapy modules may overlap or substantially overlap one another, may be staggered, or may be provided at different periods of time. Certain embodiments provide for long-term patient therapy until symptoms of PTSD have reduced and the patient has adopted exercises successful symptom management skills. In yet other embodiments, duration of therapy is under the discretion and supervision of at least one of the patient's mental health professionals. In embodiments relating to military personnel, the duration of the therapy may continue during active or reserve military service, or while any veterans' benefits are available to the patient, or as prescribed by authorized military personnel.

Embodiments of the present invention provide continuous monitoring and access to patients. In preferred embodiments, monitoring services to patients are provided 24 hours per day, 365 days per year. In certain embodiments, mental health professionals are available on an on-call basis, while educational resources and patient use of the personal device is available at all times.

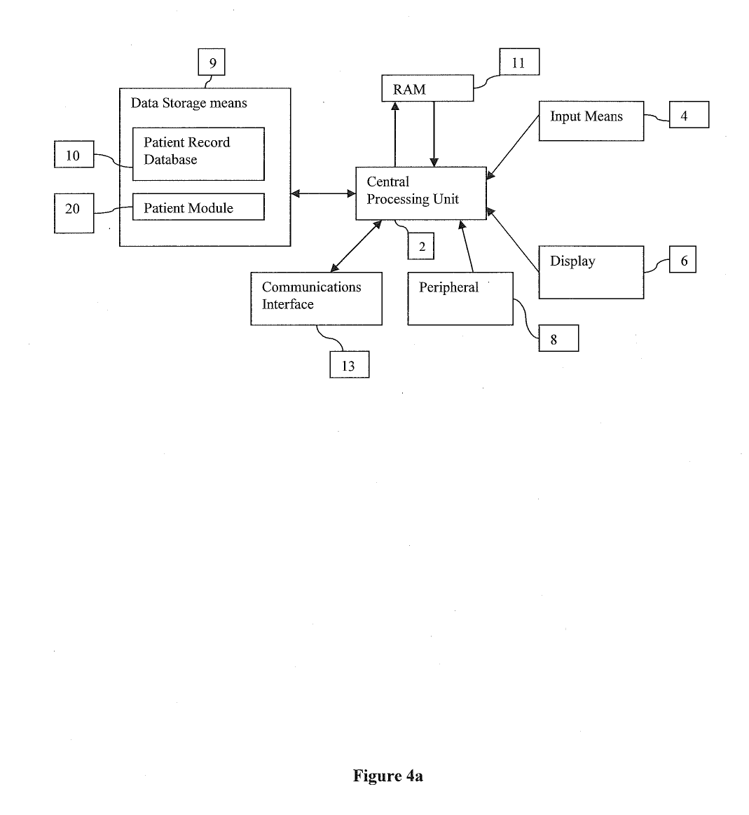

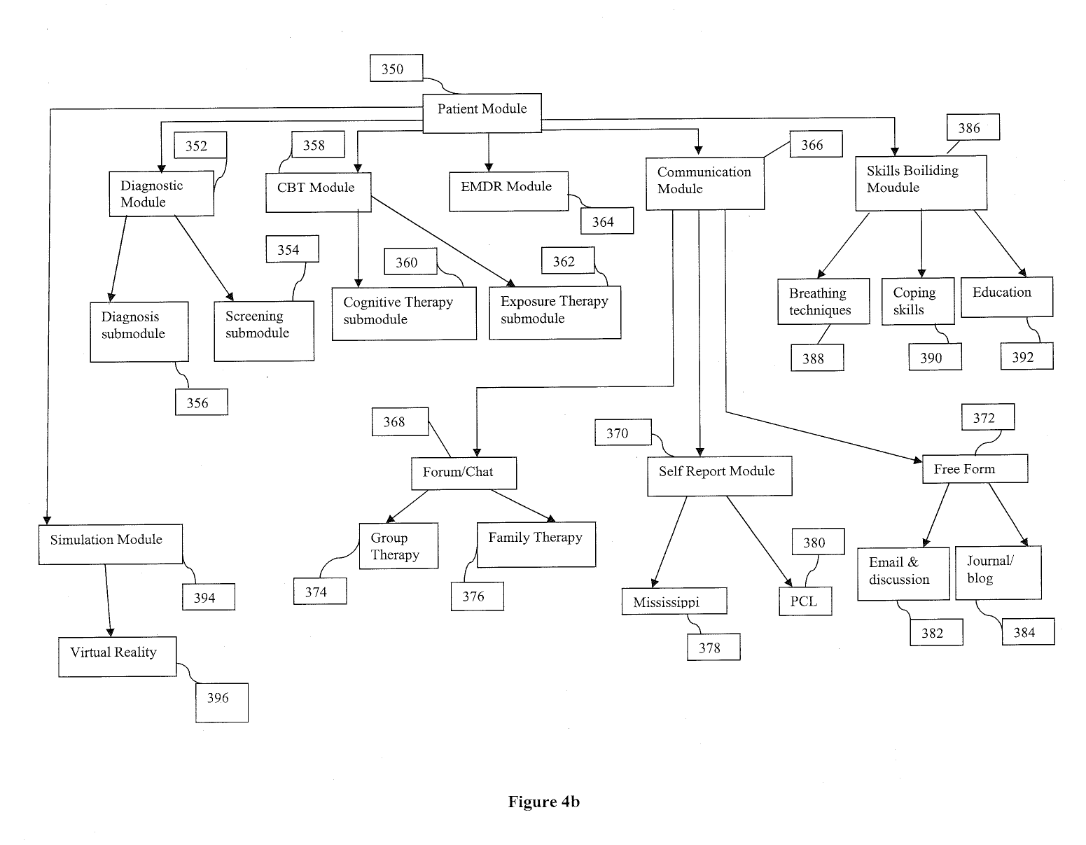

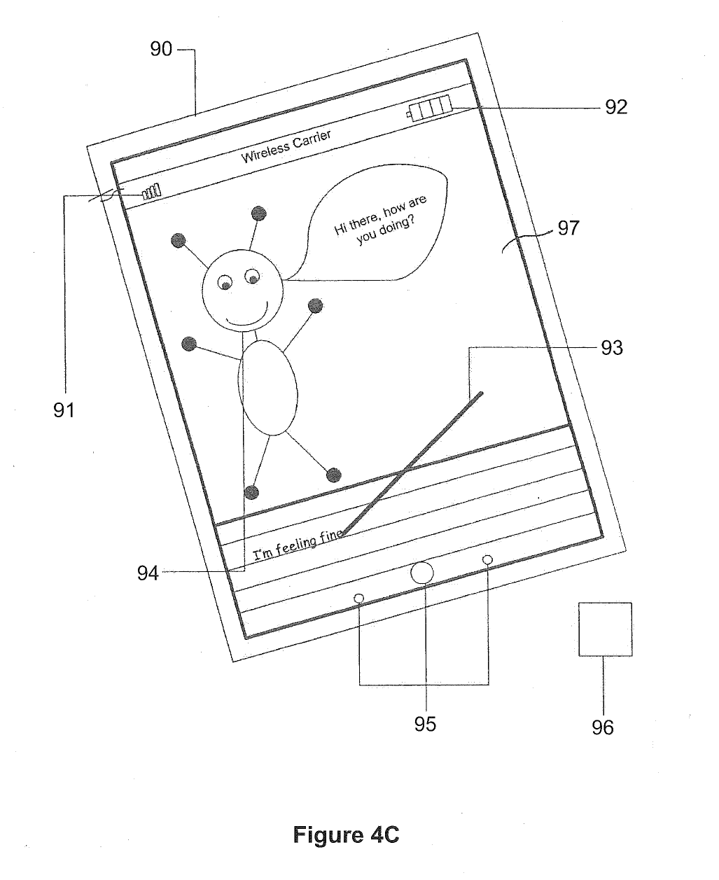

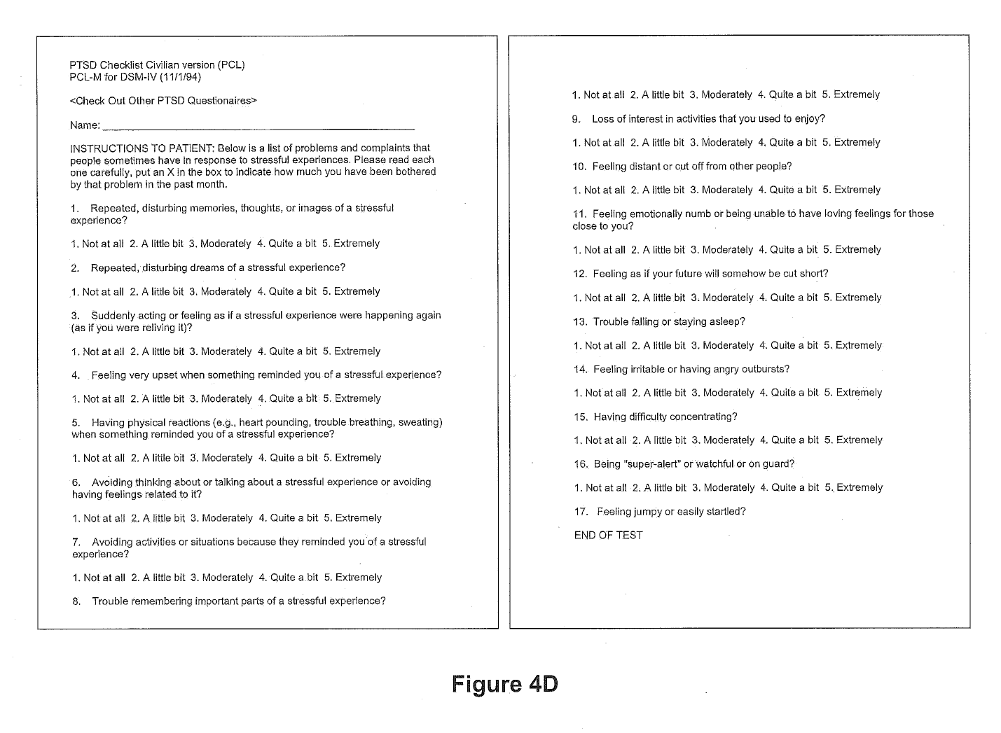

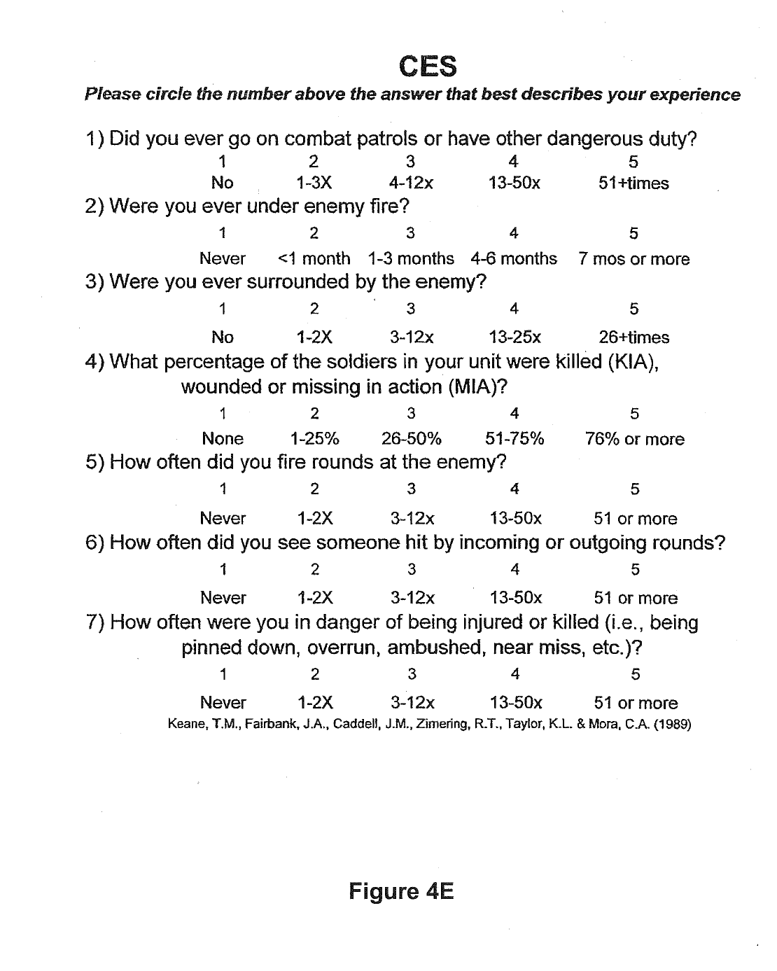

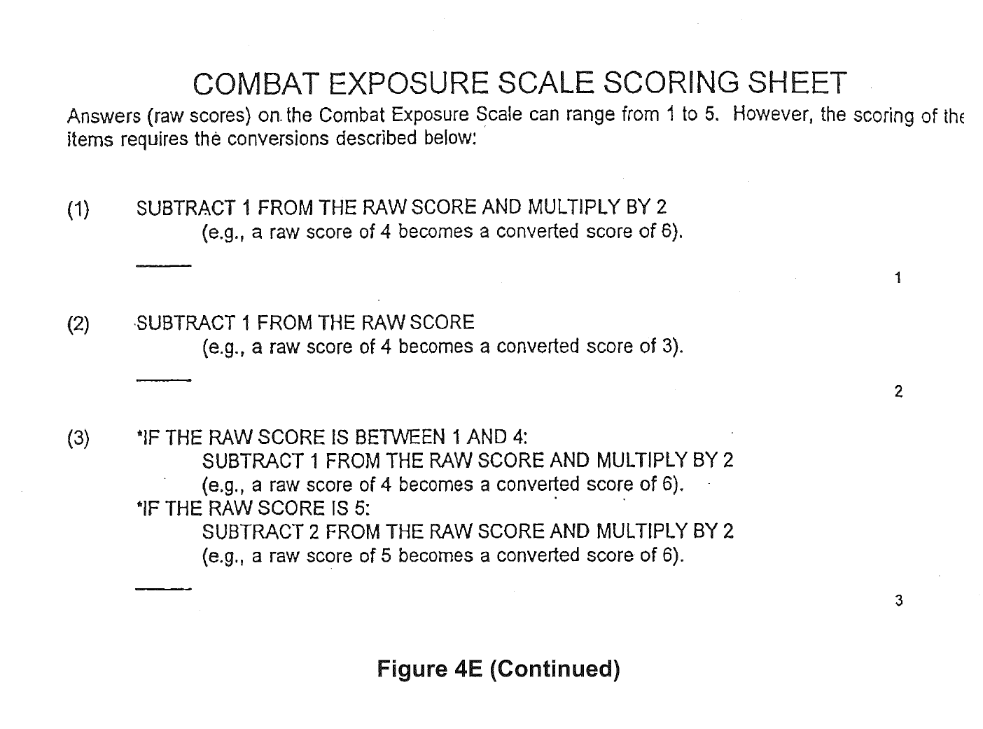

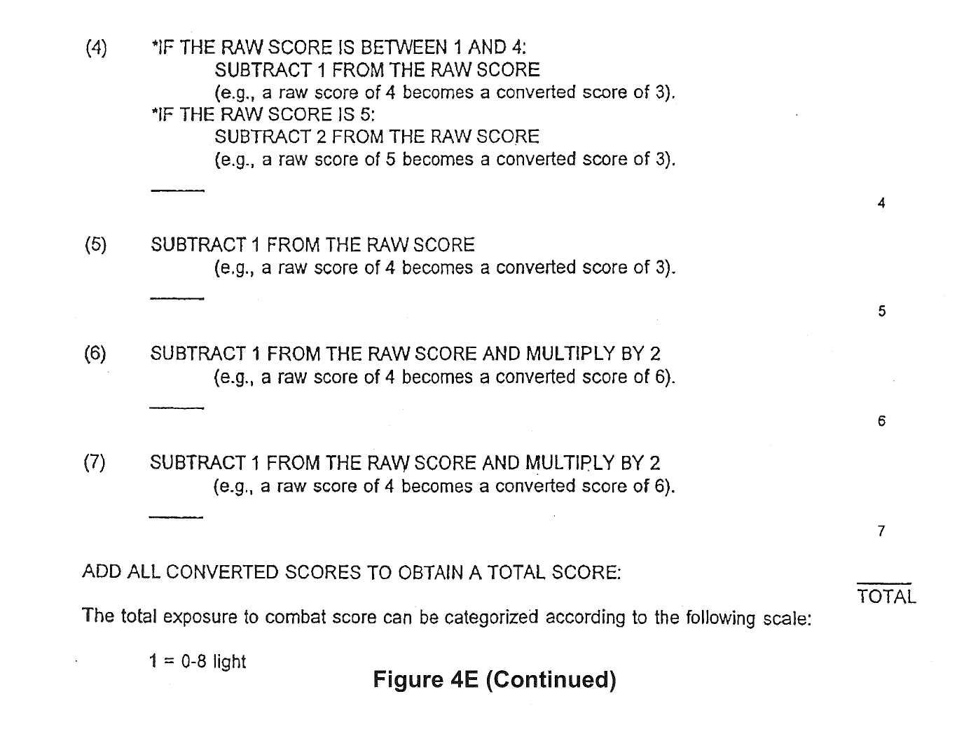

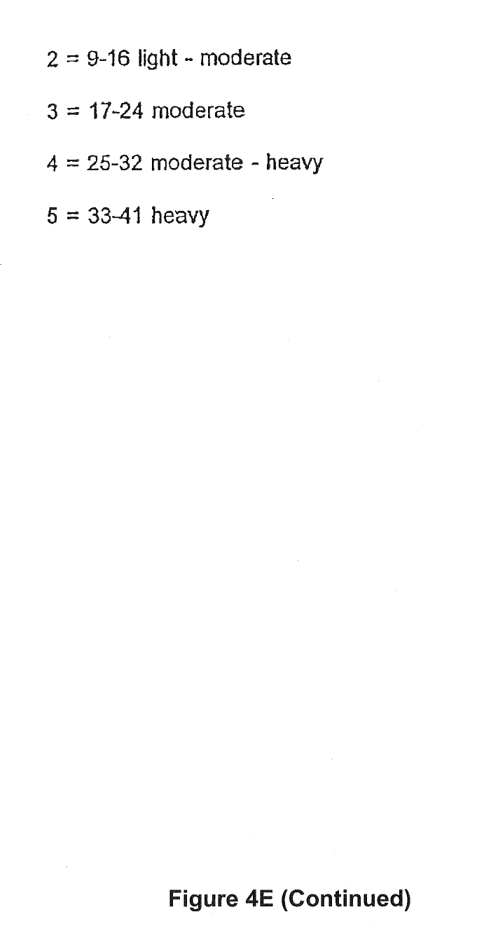

Referring to FIG. 4a, a block diagram of one electronic terminal 2 in accordance with one embodiment of the present invention is shown. The terminal 2 illustratively includes a central processing unit (CPU) 2 for controlling the operation of the terminal 2; input means 4 operatively coupled to the CPU 2, such as a keyboard, disk drives, memory storage, computer network interface, etc., or any combination thereof, for inputting patient records or module-related information into the system and for allowing a user (i.e., the patient, the mental health professional, or the central office generally) to control the operation of the system; a display 6, such as a monitor providing a graphical user interface, and a peripheral 8, which may include a printer, scanner, or system for output; data storage means 9, such as a hard disk drive or tape drive or other memory means, operatively coupled to the input means 4 and CPU 2, said data storage means 9 including an Patient Record Database 10 for storing data relating to each patient in the system of the present invention, including but not limited to, the patient's name, the other demographic or identifying information (birth date, sex, address, insurance, etc.), and patient records (including diagnoses, medications, therapy regimens and results thereof, attendance records, and other healthcare records). The data storage means 9 further includes a Patient Module 20 which includes one or more programs, processes, or instructions to be executed by the CPU 2 for administering treatments, tests, interventions or other interactive evaluation, and collecting patient data, based on, and adding to, data stored in the Patient Record Database 10. In certain embodiments, as data is collected, it is stored in the patient database 10, as shown in FIG. 4a.

Additionally, in FIG. 4a, the terminal further includes a random-access-memory 11 (“RAM”), which may be used in addition to or in combination with the data storage means 9 to store patient or treatment data as well as other dynamic data; and a communications interface 13, such as a modem or network card, which can be used to communicate with other computers and computer networks.

In practice, the terminal of the mental health professional at the central office will typically take the form of a personal computer running Microsoft Windows (Redmond, Wash.) or Apple OS (Cupertino, Calif.), but may take the form of a Unix workstation, a mainframe computer, a mid-range computer, or any other apparatus, such as the portable computing device, that can be configured to perform the functions described herein. The software described herein, including the Patient Module 20, may be written in any programming language that is compatible with the systems of the present invention.

In accordance with embodiments of the present invention, patients are provided with a terminal (interchangeably referred to herein as a “patient device”) in the form of handheld, personal electronic, preferably wireless communication and monitoring device, generally characterized as a personal computer, such as a desktop computer, or more preferably a portable electronic computer, such as laptop, notebook, netbook, or portable tablet computer, or more preferable a personal digital assistant (PDA), smart-phone, mobile phone, portable gaming platform, ultra-portable laptop, netbook computer, or the like that facilitates and permits communication with and input by patient. Exemplary embodiments of these devices include the RIM Blackberry® (RIM, Inc., Canada), Apple iPhone® and iPad® (Apple Computers, Inc., Cupertino, Calif.), Sony PlayStation Portable® (PSP) (Sony Computer Entertainment, Japan), and Nintendo DS® or DSi® (Nintendo Inc., Japan). In preferred embodiments, the handheld device is equipped with communications hardware (e.g., radio signal antennas and modems), which allows connection to one or more communication networks, wired or preferably wireless. In the most preferred embodiments, the wireless device is portable and usable anywhere in the world. In certain embodiments, the patient device may be equipped with global position satellite (UPS) technology to monitor the location of the patient.

Embodiments of the system and methods of the present invention are configured to be accessible using many different electronic devices. Preferably these devices are commonly available, and may be further configured as needed. In preferred embodiments, the electronic devices are not a specialized device, specific for mental health patients, or cannot be readily identified as medical devices, such as by label, design, or otherwise. Accordingly, general purpose electronic devices contemplated in this invention include, but are not limited to, a home personal computer, a cell phone, a personal digital assistant, etc. More preferred devices use the patient's own devices, which the patient possessed prior to mental health treatment in accordance with the system and methods of the present invention. As such, the devices of the present invention avoid situations in which patients are stigmatized for having a mental health device.

Using the communications interface of the patient device, which preferably uses a wireless communication connection, the patient device is functionally connectable to at least one mental health professional, at least one mental health agency or a mental healthcare network for preferably continual monitoring and assessment of the patient and preferably two-way communication. To communicate, the patient engages the patient device using the graphical user interface displayed on the monitor and inputting his/her response. In some variations, the patient submits the response by activating a corresponding button, such as a “submit” or “post” button. In some variations, the remotely located clinician can access the patient device. While functionalities of the patient device are substantially similar, specific operation of each device would be understood to one skilled in the art.

Communication between the patient device and the mental health professional (via the central office, as necessary) is preferably provided through one or more telecommunications or communication means, such as a cellular networks, Wi-Fi networks, Bluetooth networks, infra-red (IR) networks, or the like, and may be transmitted, for example, through SMS, MMS, landline telephone, cellular, GSM, CDMA, Wi-Fi, Wi-Max, wireless transmission, the Internet, LAN, WAN, email, and any other electronic tone, pulse, or packet transmitted via wired or wireless paths or combinations thereof.

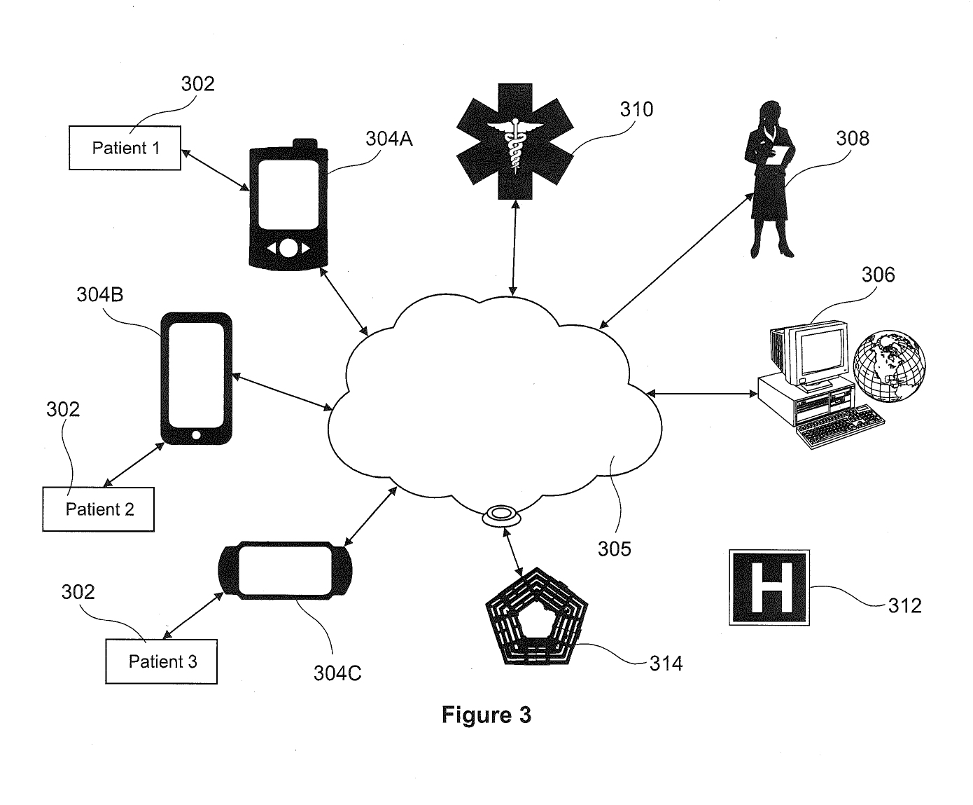

FIG. 3 provides an exemplary network diagram of the present invention. A plurality of patients 302, each of which having access to a patient device 304a, 304b, and 304c, are equipped with a communication means that allow the patient devices 304a, 304b, and 304c to communicate with an electronic public switch or preferably packet switch network 305, such as the Internet or private network. The terminal 306 represents the terminal of the mental health professional or at the central office, which would be accessible by the mental health professional 308. Accordingly, the mental health professional 308 may receive communication directly on his/her own electronic terminal 306, such as a computer or PDA through the network 305 as well. Other parties, such as health facilities 312 (e.g., a hospital), first responders 310 (e.g., EMS), and military personnel 314 may also be communicably connected to the network 305.

The patient device is configured to execute one or more computer-executable, computer-implemented programs, referred to herein as patient modules, to allow communication and mental health assessment. The program may either be pre-loaded on the patient device or may be broadcast or uploaded in real-time to the patient device at the discretion of the mental health professional. In some embodiments, modules may be added and removed from the patient device, preferably by the mental health professional only, but it is also contemplated that the modules may be added and removed by the patient. In preferred embodiments, modules are available through the communication network, such as through websites, remote servers, or computer clouds, preferably having an authentication protocol or a patient identification means, such as a registration, log in, and password. In more preferable embodiments, the availability of modules is customized by the patient. For example, the patient may have access to a prescribed set of modules, which are preferably provided sequentially or partially simultaneously, as prescribed by the mental health professional.

Embodiments of the present invention, particularly embodiments using and/or implementing modules discussed herein, may be manually done by mental health care professionals. In preferred embodiments, any aspect that is manually implemented by a mental health professional may also be automated based on algorithms, signaling technology, and sensors as needed, preferably under the supervision of, or in accordance with the methodology prescribed by, one or more mental health professionals.

Modules may be disease-specific, symptom-specific, or may be generalized tools used in the mental health arts. In preferred embodiments, the patient modules may be directed to address and/treat certain diagnoses or symptoms. Accordingly, the specific content of each module is customizable to the patient and disease, in accordance with certain embodiments. In some embodiments, the mental health professional would install the specific module(s) relating to the patient's condition. For example, a patient with PTSD may be offered, given, or prescribed one or more executable programs incorporating or having PTSD-related tests and questions for use on his/her electronic device. For example, should the patient have PTSD, the program would have PTSD-related tests and questions. One such module, known as the inquiry module would allow mental health professionals to pose specific questions and prompts to the patient. As a result, mental health professionals can target specific issues, just as if the patient were in a face-to-face setting.

FIG. 4b provides an exemplary organizational diagram of modules in one embodiment of a PTSD patient module 350. The diagnostic module 352, which includes a screening sub-s module 354 and a diagnosis sub-module 356 provides the mental health provider with an electronic process to screen and diagnose the patient, for example, for PTSD or other recognized disorder in the DSM. The cognitive-behavioral therapy (CBT) module 358 includes a cognitive therapy sub-module 360 and an exposure therapy sub-module 362 designed and adapted to treat PTSD patients.

Embodiments of therapeutic modules include prompts (e.g., questions or action items) to the user displayed through the graphical user interface on the display (i.e., a monitor or screen). The user selects or inputs a responsive entry to the prompts, which are transmitted to and processed by the central office. In some embodiments, prompts subsequent to the patient's response are selected by a mental health professional in real-time, or in advance, such as in a predetermined script. In preferred embodiments, a script of prompts is generated electronically and specific prompts are presented through signals to the patient electronic device depending on the patient's response time, substantive response, or a combination of the two. The central office may process and record various factors attributable to the patient, such as, for example, response time, length of the response, and accuracy and substance of the response.

The present invention may further include one or more modules. The eye movement desensitization and reprocessing (EMDR) module 364 is also provided to treat PTSD patients. The system may record qualitative and quantitative factors, as would be understood to one skilled in the art, providing the mental healthcare professional with clinical information regarding the patient. The simulation module 394, with a virtual reality sub-module 396, may also be provided as a treatment for patients with a mental illness. The skills-building module 386 provides patients with one or more tools and/or training to address their mental illness. This module 386 may include a breathing techniques sub module 388, a coping skills sub-module 390, and/or an educational sub-module 392.

The communications module 366 includes one or more functionalities for the patient to communicate with the mental health professional. For instance, the communication module 366 may include a free-form communication sub-module 372 with an email and discussion sub-module 382, preferably with messaging capabilities, and a journal/blog sub-module 384, and preferably real-time, web conferencing capabilities, in some embodiments. The self-report sub-module 370 includes self-awareness applications, such as the Mississippi scale sub-module 378 and the PTSD Checklist (PCL) sub-module 380. A sample PCL is provided in FIG. 4d and a sample Mississippi Scale (combat version) is provided in FIG. 4e. These sample scales provide exemplary questionnaires involved with PTSD diagnosis and treatment. Other checklists available electronically may be used and adapted for the treatment of various mental illnesses, such as PTSD

Additionally, a group therapy sub-module 374 and a family therapy sub-module 376 may be provided through a forum/chat sub-module 368, which allows interaction by two or more parties at the same time. Each of the modules disclosed may be used independently or in conjunction with one another. The function of these modules is to allow third-parties, e.g., other patients with post-traumatic stress disorder or symptoms thereof, or friends or family, to participate or observe the therapy, or to provide support to the patient. In some embodiments, these therapy modules allow at least three parties to engage in real-time, or substantially real-time, live communications, either through text, images, audio, video, or a combination thereof. For example, private chats and video-conferencing technologies may be used. In more preferred embodiments, between 3 and 6 parties, including the patient and mental health professional may communicate with one another at the same time.

In preferred embodiments, communications between the patient and mental health professional are intended to provide psychological, emotional and cognitive assessments. In some embodiments, physical measurements of the patient may be optional collected and analyzed to make additional determinations and diagnoses of the mental illness and/or its symptoms. In embodiments of the invention, the communication between the patient and mental health professional can take any form, from general, free-form discussions, such as through messaging or narratives composed by the patient, to preferably structured clinical assessments, such as forms or questions and answers, which requires responses to direct requests. Preferred communication forms include those that promote or require interaction between the mental health professional and the patient. It would be understood by one skilled in the art that peer-reviewed or generally accepted forms and questionnaires in the mental health field may be adapted for use in the modules. In certain embodiments of the present invention, mental health professionals and patients can engage in real-time or substantially real-time discussions via email, text or instant messaging, electronic chatting, interactions in online forums, or through telephonic communication. In preferred embodiments of the present inventions, patients can maintain an electronic journal or blog, at a time interval (e.g., daily, weekly, etc.) determined by the mental health professional, on one or more topics, which are preferably predetermined by mental health professionals or provided during treatment. One topic may include a self-assessment in which a patient describes his/her mood, anxiety level, and overall self-perception, for example. In other embodiments, the mental health professional may provide electronically available (e.g., online) homework assignments to be completed by the patient and reviewed by the mental health professional.

More focused or structured communication between the patient and the mental health professional may take the form of individual and group psychotherapy, psycho-educational interventions, cognitive testing, skill building and training, and general psychological and/or psychiatric evaluation, each of which may be offered to the patient in the form of a module on the patient device. Preferably, the system of assessment of the patient may include artificial intelligence or similar processing to dynamically present relevant or appropriate questions related to the patient's affliction, mental or physical state, and/or to environmental circumstances. For example, the mental health professional may electronically administer one or more psychological tests or activities to assess the patient's mental health. In some embodiments, the assessment of the tests/activities may be conducted during the test, and may for example, cause the selection or de-selection of certain questions or follow up tests. Additionally, in some embodiments, the mental health professional may provide virtual reality simulations, or tools and information for disease management, such as websites directed to understanding PTSD and/or breathing exercises, for example.

In preferred embodiments of the present invention, patients are provided with a self-reporting feedback mechanism and question and answer programs, wherein the mental health professional provides one or more questions that are to be answered by the patient. The self-reporting module generally is believed to enhance the efficacy of treatment by allowing patients to track their progress and link their skills practice with overall success in managing symptoms associated with PTSD and other traumatic stress or co-occurring disorders. In preferred embodiments, the questions are directed to determining the extent of PTSD or symptoms thereof and patient health with respect to diagnosis and/or treatment of PTSD. The communication between the patient and the mental health professional may be generalized so as to be applicable to more than one patient or may be customized per individual patient.

These communications may be transmitted at regular intervals, such as, for example, multiple times per day, once per day, two to four times per week, two to twelve times per month, once per month, or some other time period, or irregular basis, such once every few weeks, or as deemed appropriate by the mental health professional, by other personnel interested in the health of the patient, by the patient satisfying certain predetermined threshold levels, or a combination of the three. In some embodiments, the communications may be “on-demand,” i.e., as required by the patient, based for example on a patient's clinical evaluation, a patient's physical or psychological state, or at the determination of the mental health professional, in which case the patient may initiate contact with the mental health professional or vice versa, as necessary. In certain embodiments, the frequency and/or regularity of the communication may be escalated or deescalated in accordance with a predetermined therapy regimen, manifestation of symptoms, or the professional discretion of the mental health professional.

In preferred embodiments of the invention, there is interaction between the modules. For example, a patient's response or input in one module may require the use of another module. In some embodiments, a patient's response or input in one module may stimulate the presentation of another module to the patient. In most preferred embodiments, the interaction between modules is predetermined through an automated program. In some embodiments, patient input in one module stimulates a mental health professional to provide another module to the patient. Some embodiments of the invention provide a graphical user interface (GUI) to the patient that provides the user with finite number of executable options.

In accordance with preferred embodiments of the invention, the patient would be required to complete a therapy module or assignments or respond to inquiries from the mental health professional as often as prescribed by the mental health professional or at regular intervals such as daily, bi-daily, or weekly. As would be understood to one skilled in the art, patient assignments and the frequency thereof, may be dictated by the patient's mental illness and prescribed treatment regimen.

In certain embodiments, to ensure compliance with the mental health therapy, and to ensure accuracy and truthfulness of patient responses, mental health professionals may implement a regular or highly-regular interval in which a patient responds to messages received or signals sent to the electronic device. This would ensure that the patient is at least responsive. Moreover, data received from the patient may be preliminarily reviewed by the mental health professional, or a computer-implemented processor, which, based on a executable program or algorithm, screens variability in patient responses and which may quickly and efficiently detect inaccuracies or untruthfulness in patient responses, or substantive changes in the patient's mental health status.

Embodiments of the present invention are provided to a population of patients prior to determining whether patient's have the psychological disorder, such as PTSD, or are exhibiting symptoms of a mental illness that has not yet been diagnosed. For example, military personnel may be provided with the system and method of the present invention upon return from combat as a matter of protocol. In such populations, embodiments of the invention may be used to detect the scope of symptoms, and in some variations, transition from a first set of symptoms to a second set of symptoms. Thus, this embodiment of the invention may detect onset of the disease or appearance of symptoms as they arise. Preferred embodiments include modules responsive to the onset of a disease or symptoms, such that modules are provided to the patient as disease and symptoms are detected. Preferably, this operation is automated by an algorithm. These embodiments can also vary treatment based on transition of symptoms. Accordingly, embodiments store, preferably electronically, a range of symptoms and a range of corresponding treatments, and can provide the appropriate treatments as needed by the patient.

The system and methods of the present invention provide an interactive platform that facilitates psychological therapy and skills building and training in a remote environment. Thus, the system includes at least one therapeutic module and program (i.e., electronic, computer-implemented instructions or executable programs) or processes providing elements of the psychological therapy and/or skills building exercises, as would be understood by one skilled in the art. Each program or process may include features that allow the mental health professional to interact with the patient, for example, to solicit patient input, activity, and/or response. In the most preferred embodiments, the programs use artificial intelligence to dynamically display questions to analyze the patient's mental status. Modules may be used alone, or in combination, in embodiments. In some embodiments, modules are manually implemented by a mental health professional. In preferred embodiments, modules are automated.

In some embodiments, the screening module will include software and processes to allow the mental health professional to identify symptom clusters, using a decision tree format, preferably providing inquiries specific to particular mental illnesses, such as PTSD.

Therapies provided through the present invention may be used alone or preferably in conjunction with traditional, face-to-face psychological therapy and/or medications, such as selective serotonin reuptake inhibitors (SSRIs), other anxiolytics, or medications known in the art to treat PTSD. In preferred embodiments of the present invention, one or more therapy modules are included on the patient device and on the corresponding healthcare worker terminal or at the central office. These therapy modules provided to the patient include conventional psychological therapies adapted for remote administration.

Generally, diagnosis of the mental illness via assessment of psychiatric or psychological symptoms, related problems, and factors influencing functioning, is a preliminary, if not initial clinical step, in mental illness cases. This step is preferably conducted in-person, but may also be conducted remotely through electronic diagnostic and assessment tools in the diagnosis module in the present invention. Diagnostic questions and inquiries may be adapted for electronic administration.

In certain embodiments, for PTSD in particular, diagnostic and assessment tools follow or at least relate to Criteria A-F, which are outlined in the DSM-IV, for example, as six clinically accepted criteria for the diagnosis for PTSD. Accordingly, in these embodiments, the diagnosis module provides a system and method for information-gathering related to eliciting one or more of the following: 1) a traumatic event encountered or confronted by the patient [Criterion A]; 2) the re-experiencing symptoms, such as memories, nightmares, and/or flashbacks [Criterion B]; 3) avoidance symptoms used by the patient to distance or remove himself from the traumatic event or memory thereof [Criterion C]; 4) hyperarousal symptoms which may characterize the patient's mental or psycho-physiological state [Criterion D]; 5) a duration of symptoms (e.g., preferably 6 months or more in duration) [Criterion E]; and 6) negative effects of trauma on the patient's life social and occupational functioning [Criterion F].

At least one therapy provided is specific to, or identified by a person having skill in the art as pertinent to, the patient's diagnosed mental illness. For PTSD, for instance, patients are provided with forms of cognitive-behavioral therapy, which may include cognitive therapy and exposure therapies, such as cognitive processing therapy or prolonged exposure, whereby patients are exposed to stimuli associated with their trauma (written or recorded) with the clinical goal of habituation to the traumatic stimulus and reduction of negative affective, physiological and behavioral symptom structures associated with PTSD. In preferred embodiments of the present invention, cognitive and behavioral therapies (CBT) are focused on military-related experiences or trauma, including trauma experienced in combat or as a result of other military associated traumas such as military sexual trauma (MST). In some embodiments, therapies for PTSD include programs and/or processes to administer EMDR.

Some embodiments of the system and methods of the present invention designed for treatment and monitoring of PTSD include a cognitive therapy module, adapted to be computer-implemented. In some instances, programs administering cognitive therapy provide instruction and inquiries that assist the patient to understand and reframe memories associated with the trauma that elicited PTSD. This process entails identifying thought processes identified as cognitions which may be aberrant or inaccurate. One goal of this model is to restructure or challenge associated inaccurate thoughts. Another goal is to correct or modify these thoughts thereby impacting the way people experience their lives and thus changing how they feel. Frequently this model is paired with behavioral strategies identified under the general clinical category of CBT, as would be understood to one skilled in the art.

In some embodiments, the patient is administered psychological tests and interventions that help the patient understand how certain thoughts about the trauma cause stress and exacerbate symptoms. Furthermore, therapy is generally provided to help the patient replace or mitigate thoughts of the trauma with more accurate and less distressing thoughts. These programs additionally assist patients with ways to cope with feelings such as anger, guilt, and fear. In some preferred embodiments, the patient is provided with programs that short circuit the avoidance process by bringing PTSD symptoms and treatment into the patient's consciousness.

Preferred embodiments also include programs to provide exposure therapy remotely. Research suggests that exposure therapies such as cognitive processing therapy (CPT) and prolonged exposure (PE) are the preferred modes of treatment for PTSD. These models variably utilize stimuli associated with the traumas in the forms of recounting or retellings either written or orally to achieve a clinical phenomena known as habituation. This process is identified by a decrease in subjective distress and associated avoidance from feared stimuli related to a trauma. One goal is to reduce distress and avoidance and increase a patient's ability to successfully engage in chosen activities that may have been previously feared or avoided.

In one embodiment of the present invention, for a patient with PTSD especially, the patient device includes questions, such as those about the patient's dreams and any sudden flashbacks that the patient may have encountered which bring back memories of extreme stress, or the patient's emotional status at the moment.

Some embodiments of the present invention provide programs and processes related to EMDR to change the patient's reaction to memories. These programs generally include functionalities, such as on the graphical user interface, to simulate conventional EMDR, including distractions, such as promoting eye movements, hand taps, and sounds.

While preferred embodiments of the invention contemplate a bi-directional model between the patient and the mental health professional, certain embodiments of the system and methods of the present invention may provide a multi-directional, group therapy module. Accordingly, the patient device allows interaction with at least two other parties, such as in a real-time chat room or forum. The input is designed to allow each member of the group therapy to interject, view or review peer commentary. In some forms of remote group therapy, the mental health professional facilitates the communication by providing topics or lines of discussion, or remains a silent or substantially silent observer while patients in the group therapy input comments, such as regarding issues concerning exposure and the related trauma. In yet additional embodiments, the group therapy program provides social community aspects to allow patients to build relationships with one another and to share emotions, such as shame, guilt, anger, rage, and fear.

In certain embodiments having a family therapy module, the patient, the mental health professional, and at least one family member, i.e., a concerned significant other (CSO), of the patient are linked via a remote communication network to provide functionalities similar to the group therapy module, namely group discussion and interaction, and tools for distribution and dissemination of information about the mental illness.

Embodiments of the present invention may additionally include a psychodynamic psychotherapy module, wherein the patient can be presented with techniques and problems to develop ways of dealing with emotional conflicts caused by the trauma. Aspects of this therapy are directed to relating the patient's past with the patient's present emotional and mental state. Programs of this module may include questions and prompts helping the patient identify triggers of the stressful memories and other symptoms and instructions and training to assist the patient cope with intense feelings about the past. Furthermore, this therapy module may include therapeutic functionalities, such as questions or games, to allow the patient to increase self-awareness, self-confidence and self-esteem. In certain variations of the present invention, treatments of PTSD include behavioral therapies such as acceptance and commitment therapy, as would be understood to one skilled in the art.

Particular embodiments of the present invention also include a skills building and training module. The features of this module, which may be distinct from or the same as features provided in other therapy modules, may include programs and processes to provide the patient with a basic, intermediate, and advanced education regarding PTSD, symptom management, as well as coping and stress management. In some embodiments, patients are provided with an educational module that connects patients to electronically accessibly informational resources, such as psycho-education through websites and other audio and/or visual segments.

In preferred embodiments of the invention, the therapy and skills building module includes one, preferably two, and more preferably all three, electronically adapted versions of prolonged exposure cognitive processing therapy, a PCL, and the Mississippi Scale for PTSD, particularly the combat version.

Embodiments of the present invention may implement projective testing. In one exemplary embodiment of a software-based or computer-implemented program of one or modules, the software asks the patient to select a character that will guide the patient through the module. Through the character, the patient is asked a series of questions that would quantitatively assess mental status, such as the patient's emotional state at that point in time. In this embodiment, the character may also request a journal entry to present a qualitative description of the patient's current status. This journal entry may be transmitted to the mental health professional so that the content may be analyzed.

Aspects of this exemplary embodiment may include a test that requests the patient to draw an image which accurately displays his/her mood. This image or data related to keystrokes or patient input will also be transmitted to the mental health professional to facilitate qualitative assessment of the patient's mental and emotional status. In certain embodiments, patient results from the administered test would be transmitted to an electronic database to store the information and computer implemented processor, to analyze the information. In certain embodiments, the processed information may be provided to the mental health professional responsible for care of the patient, thus allowing the worker to assess the condition of the patient or to the proper personnel in the case of an emergency. In FIG. 4c, an exemplary patient device 90 and an exemplary graphical user interface 97 of a patient handheld device 90 is presented. The patient device 90 includes controls 95 and an input device 93 (e.g., stylus). The interface 97 displays a character 94 to guide the patient through assignments and exercises. The interface 97 also shows is the patient the strength of the signal 91 to the communication network and the battery life 92. This exemplary embodiment of the handheld device 90 includes a removable memory cartridge 96, which would provide an executable module.

In some embodiments of the present invention, an alert module allows the mental health professional to contact clinical staff or emergency services on the patient's behalf, such as if symptoms worsen, or if the patient is at risk (e.g., an increase in risk factors associated with suicide). As used herein, an “alert” encompasses any communication or notification between two or more parties, preferably the mental health professional and one or more first responders, such as EMS. The alert may be transmitted through any method of communications understood by one skilled in the art, including, for example, an audible signal (e.g., a siren), a voice call, SMS, MMS, cellular, GSM, CDMA, Wi-Fi, Wi-Max, wireless transmission, the Internet, LAN, WAN, email, and any other electronic tone, pulse, or packet transmitted via wired or wireless paths or combinations thereof. The alert is in real-time or substantially in real-time in preferred embodiments.

In some embodiments, data collected from the patient module is collected and stored. Collected data may be stored or associated with the patient profile. Thus, if a patient relocates, data may be forwarded to his/her future mental health professional. Embodiments of the invention provide a novel ability to track and monitor patient outcomes via instantly collected and aggregated data, including patient demographics, symptom presentation, and patient compliance and participation in therapy, for example.

Embodiments of the present invention preferably meets any regulatory standards (e.g., HIPAA) applicable to the healthcare setting by implementing security and privacy provisions throughout the system to maintain confidentiality and to avoid compromise of private and/or personal information. In preferred embodiments, the level of security and privacy at least meets those prescribed by the U.S. Department of Defense or other regulatory body for military personnel. In certain embodiments, the present invention meets the standards prescribed in the current Federal Information Processing Standards Publication (“FIPS”).

In many embodiments, communication between the patient and the mental health provider are encrypted at least 64 bits, preferably 128 bits, and more preferably 256 bits. In other embodiments, data is transmitted through secure networks. In preferred embodiments, access to patient data is provided with a authorized log in, password, personal identification numbers (PINs) and the like, and in some instances using site keys, as used in the banking industry, as would be understood to one skilled in the art. In some embodiments, data stored on the patient device may be detected remotely by authorized personnel.

As used herein, “real-time” is preferably defined as instantaneous. In the most preferred embodiments, real-time connotes transmission and receipt of information electronically in within seconds or minutes of sending the information. As used herein “substantially real-time” is related to receiving transmissions within days, preferably within hours, more preferably within 30 minutes, even more preferably within 15 minutes, and most preferably within 5 minutes from the time the message was originally sent. Real-time and substantially real-time also encompass a time removed from the current time, wherein a message is received a quarter minute, a half minute, a minute, two minutes, or more from the time the message was sent. In some embodiments, near real-time is defined as a time occurring in the past.

Embodiments of the present invention are preferably directed to PTSD. However, embodiments of the present invention may be directed to other mental illnesses. Modules may be mix and matched as required for treatment of the mental illness and/or as prescribed by the mental health professional.

Exemplary Systems and Methods of the Present Invention

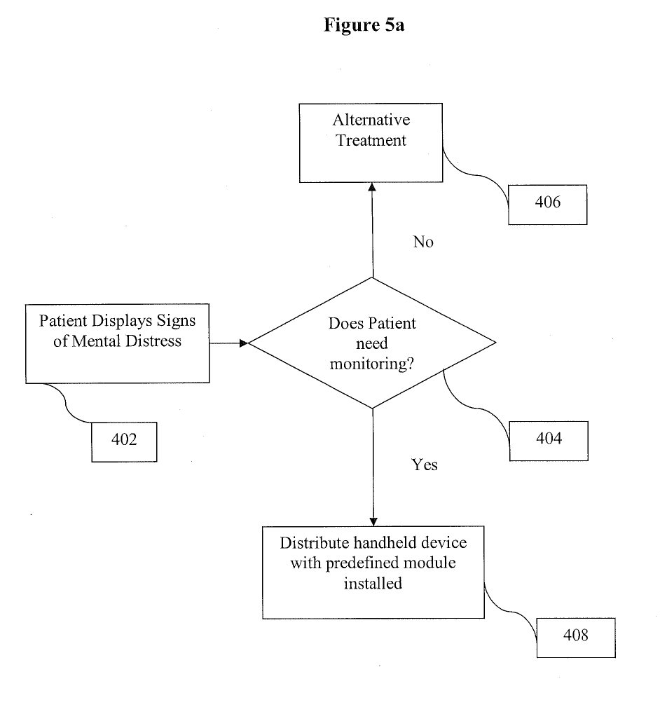

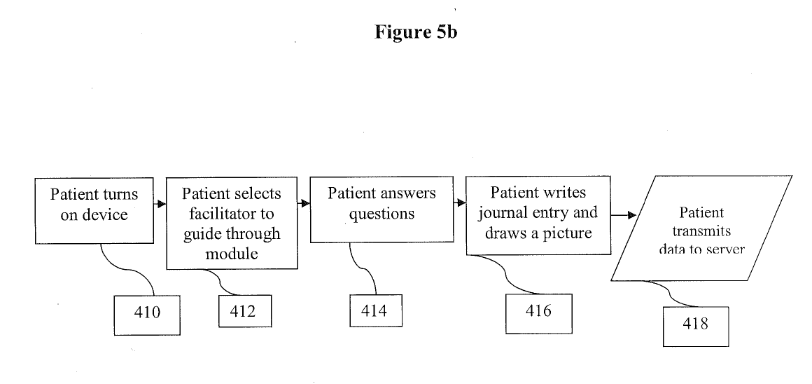

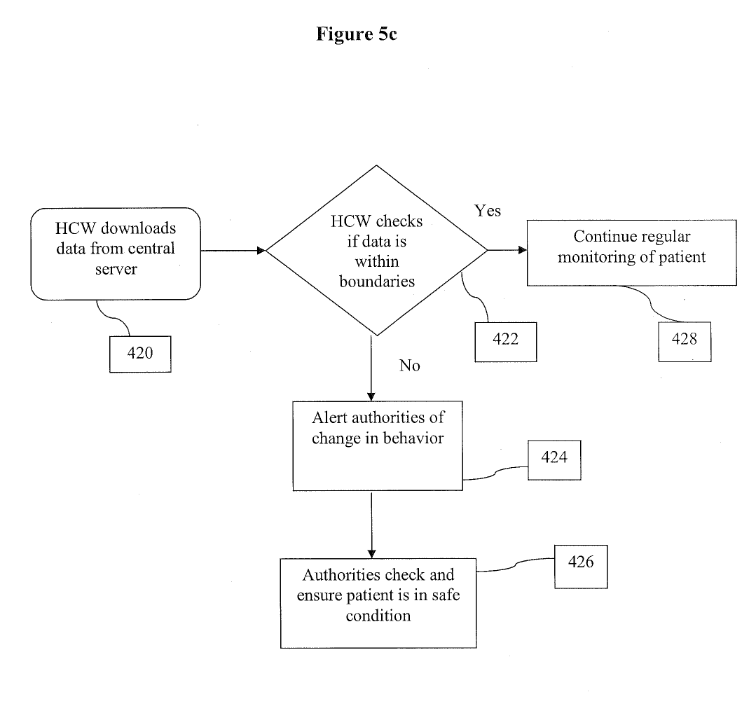

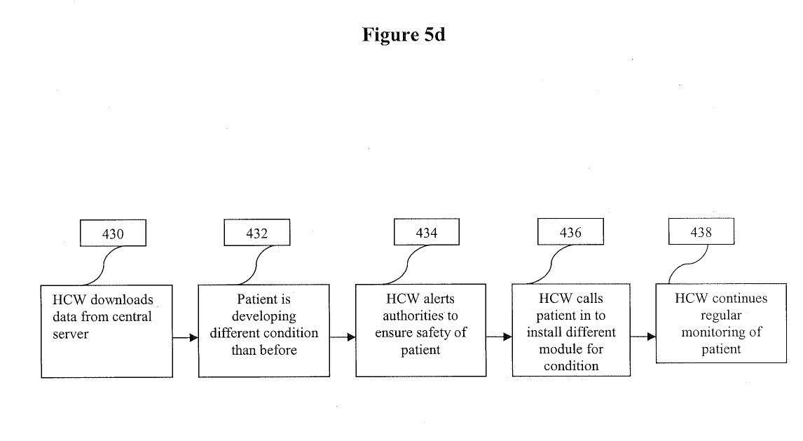

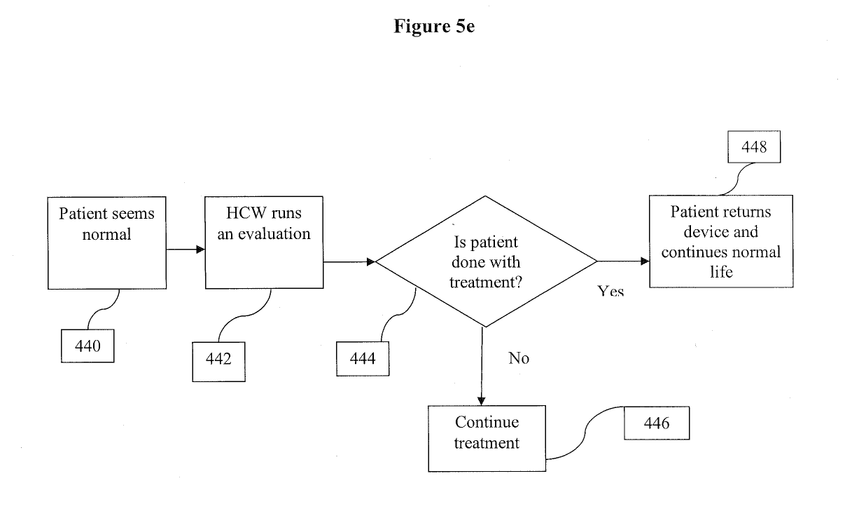

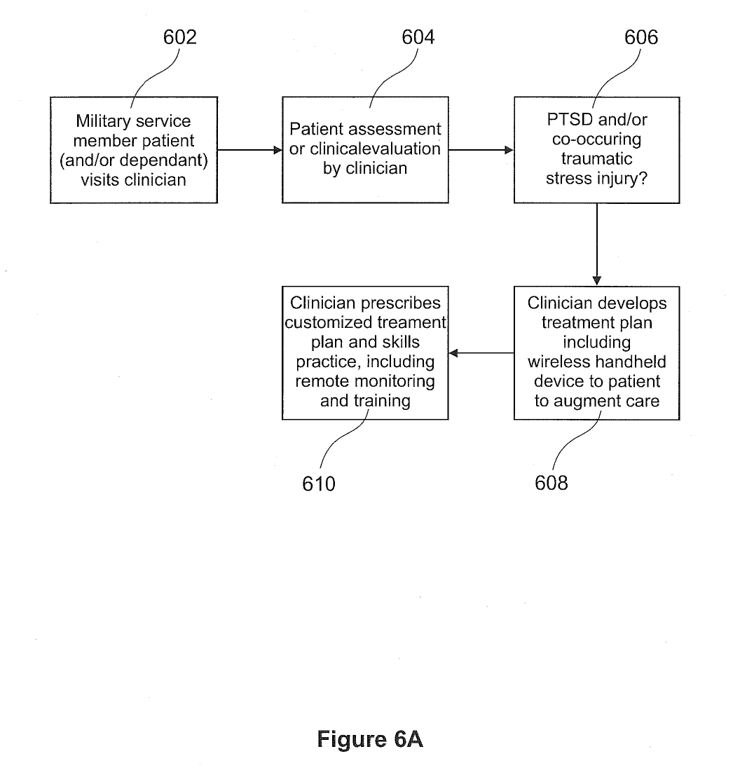

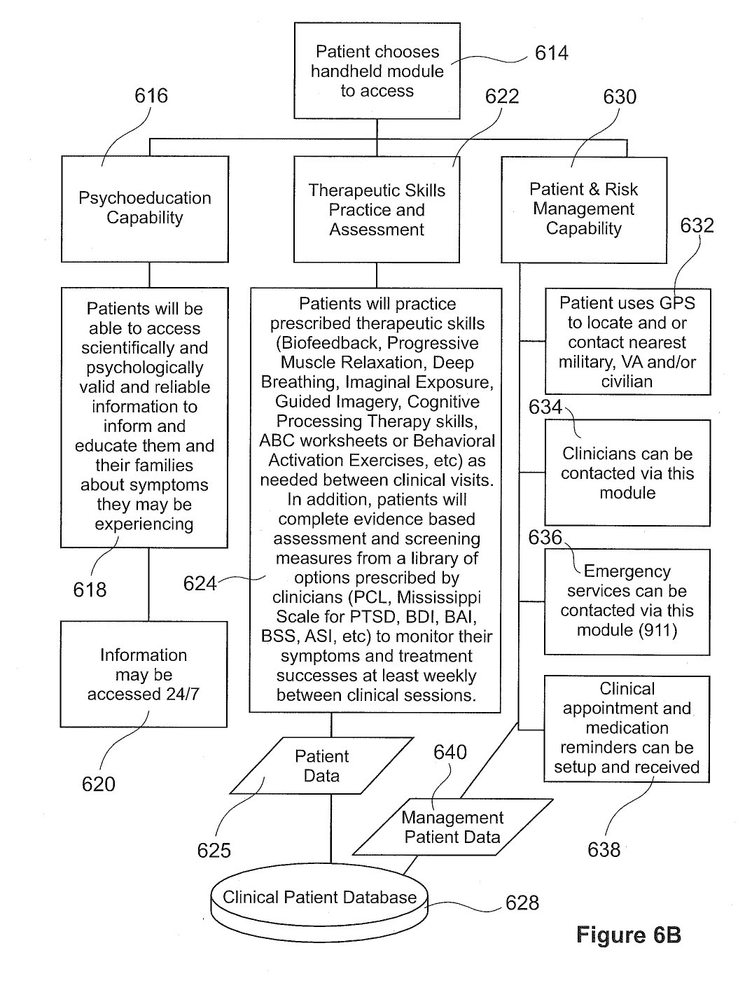

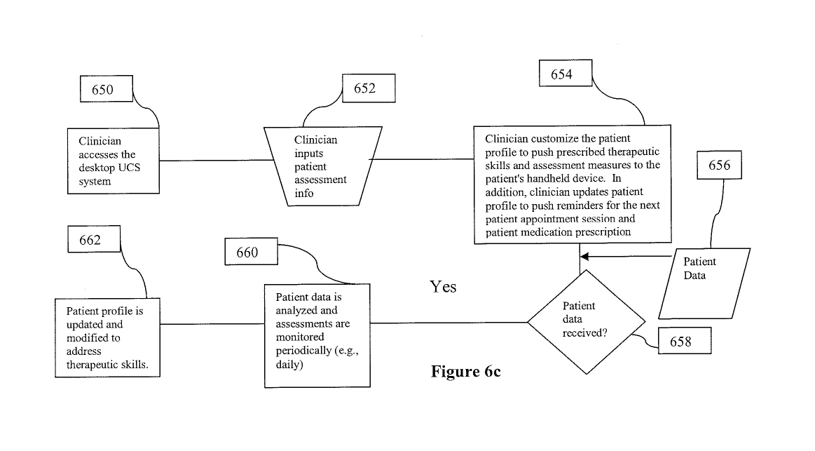

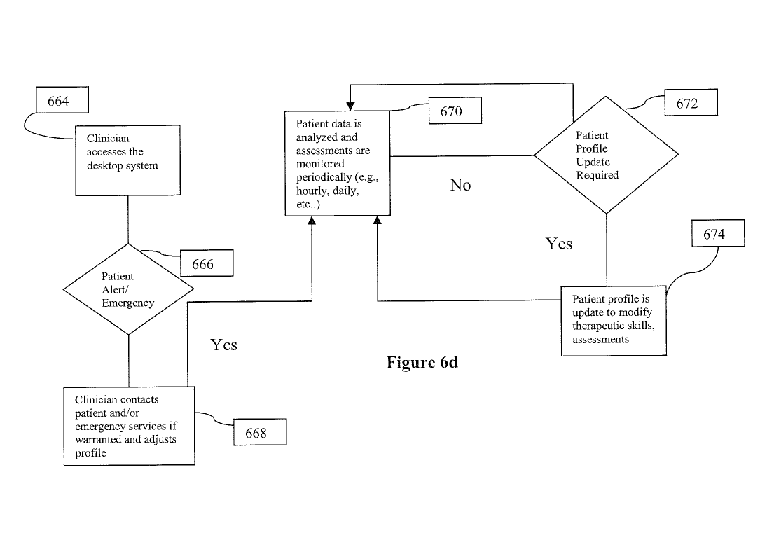

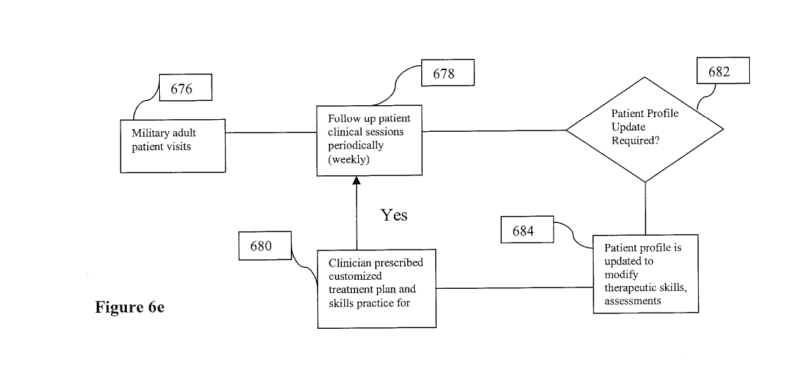

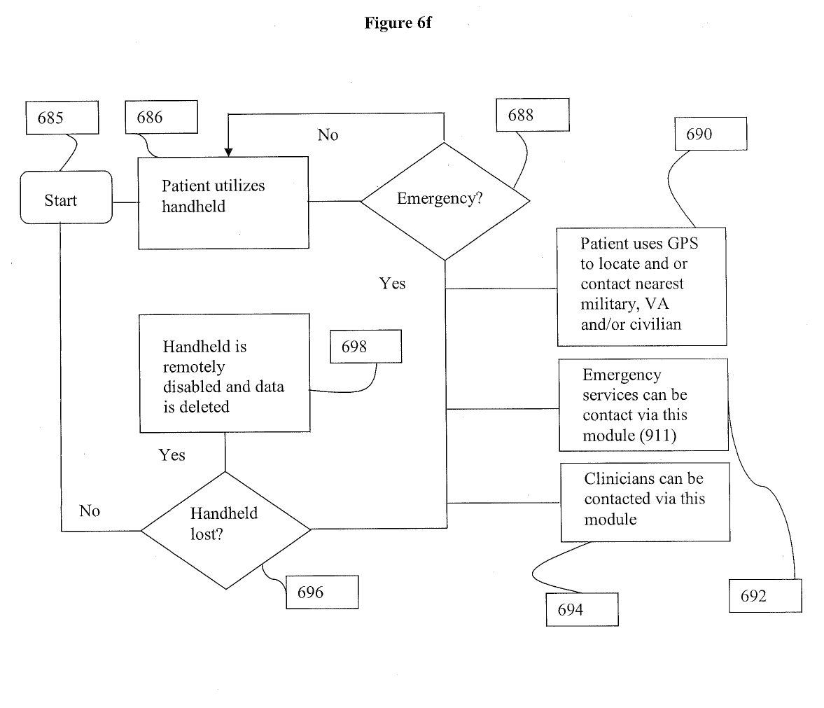

Exemplary methods of the present invention are represented in FIGS. 5a-e and FIG. 6.

In FIG. 5a, in step 402, a patient displays signs of mental distress and seeks diagnosis from a mental health professional. In step 404, the mental health professional determines whether the patient requires monitoring, in accordance with the system and methods of the present invention. In one alternative, the patient does not require remote monitoring, as shown in step 406. As shown in step 408, the patient is chosen for remote, electronic monitoring and is provided with a patient device having preinstalled patient modules, as prescribed determined by the mental health professional.

In FIG. 5b, as shown in step 410, the patient activates the portable, handheld device. Using the graphical user interface, as shown in step 412, the patient selects a character facilitator to guide the patient through the required therapy module. In response to one or more questions in the module, the patient inputs his/her response, as shown in step 414. Additionally, this embodiment includes a series of projective tests, in which the patient writes a journal entry and draws a self-awareness picture, as required by the module, as shown in step 416. Upon completion of the module, the patient transmits the data to the central office or directly to the mental health professional, as shown in step 418.

In FIG. 5c, the mental health professional has received the patient's completed therapy module and downloads it to or accesses it through his or her personal electronic device (e.g., a personal computer), as shown in step 420. The mental health professional initially checks the patient's responses which yield important data about mental health functioning and status, step 422, and if the patient shows an elevated, at-risk profile, the mental health professional may contact the patient and take needed steps to increase overall safety, and/or to the extent permitted by law or by consent of the patient, may alert authorities of the change in behavior, as shown in step 424. Authorities, such first responders or even the mental health professional, ensure the mental healthcare of the patient, preferably in-person or over the phone if necessary, as provided in step 426. If the patient manifests an elevated at-risk profile, the mental health professional continues regular monitoring of the patient, as shown in step 428.

Alternatively, in FIG. 5d, after the mental health professional obtains a copy of the patient's responses, as shown in step 430, an analysis of the responses indicates that the patient is suffering from different condition than the condition in the original diagnosis, as in step 432. The mental health professional may contact first response authorities if necessary, as in step 343, and request face-to-face meeting to install a different module, preferably a module specific for the new condition, as shown in step 436. It should be noted that new modules may be posted to the remote user via communication networks, in some embodiments. Under each newly posted module, the s patient would continue treatment and the mental health professional would continue to monitor the patient at regular intervals, as shown in step 438.

In FIG. 5e, the mental health professional determines whether ongoing treatment is still required in a patient who appears to be free of symptoms of the mental illness, as shown in step 440. The mental health professional conducts an in-person or remote psychological assessment, as shown in step 442, and determines whether the patient requires additional treatment, as in step 444. If the patient does not require additional treatment, the patient returns the patient device, or therapy modules installed thereon, as shown in step 448. If the patient requires additional treatment, the patient remains under the remote psychological system, as shown in step 446.

In FIG. 6a, in step 602, a military service member visits a mental health professional for an in-person consultation. As shown in step 604, the mental health professional conducts a clinical evaluation, and determines symptoms of PTSD in the patient, as shown in step 606. The clinician then develops a customized treatment plan, which includes remote treatment using a patient device, as shown in step 608. As prescribed by the mental health professional, the customized treatment includes skills practice and training, as well as remote monitoring, as shown in step 610.

In FIG. 6b, in step 614, a patient accesses the modules on the patient device, which includes a psych-education module, a therapy and skills practice module, and a risk management module, as shown in steps 616, 622, and 630 respectively. In the psycho-education module, shown in 616, the patients may access information, including facts and research, regarding PTSD, as shown in 618. This information is available at any time, as shown in 620. In the therapy and skills practice modules, shown in 622, patients will practice prescribed treatment techniques, exercises, and practices, including biofeedback, progressive muscle relaxation, deep breathing, imaginal exposure, and skills associated with prolonged exposure and cognitive processing therapies, ABC worksheets, and behavioral activation exercises, as shown in step 624. Additionally, patients can complete evidence-based assessments and screening measures, such as responding to scales and checklists, as also shown in step 624. As shown in steps 625 and 628, respectively, patient data is collected in this module, and stored in patient database.

For the risk management module of step 630, the systems and methods provide means to determine the location of the patient through GPS technology, as shown in step 632. Additionally, other clinicians and emergency personnel may be contacted, as shown in steps 634 and 636, respectively. A scheduling component allows scheduling of appointments and medication, as shown in step 638. Patient data may also be collected and stored, as shown in steps 640 and 628.

As shown in FIG. 6c, the mental health professional can monitor the patient using patient data, which is received electronically on the computer, as shown in step 650, and assess the patient's mental health profile, as shown in step 652. Based on the analysis, the mental health professional may make changes to the patient's therapy regimen, and may accordingly adjust the electronic modules provided to the patient, as shown in 654. For example, the mental health professional can push new programming, and other treatment modifications, such as new consultation appointments, to the patient. As collectively shown in steps 656, 658, 660, and 662, is the mental health professional conducts a similar analysis each time new patient data is received and analyzed.

As collectively shown in steps 664, 666, 668, 670, 672, and 674 of FIG. 6d, the mental health professional may determine that patient data reveals a patient at-risk situation, and if necessary, the mental health professional may contact emergency services. As subsequent patient data is received, the patient profile may be updated and therapies may be adjusted accordingly.

In the exemplary embodiment collectively shown in steps 676, 678, 680, 682, and 684 of FIG. 6e, a military adult patient enters psychological therapy, first in weekly, in-person sessions, followed by adjustment to remote monitoring based on changes in the patient's profile and, implementation of new therapies, as determined by the mental health professional.

As collectively shown in steps 686, 688, 690, 692, 694, 696, and 698 of FIG. 6f, in an emergency, the handheld patient device allows a user to instantly communicate with a first responder, a healthcare facility, like a hospital, or one or more mental health professionals. In the event the handheld patient device is lost, misplaced or stolen, data is encrypted to prevent access by a third-party. Moreover, data in the device may be deleted remotely by the central office or mental health professional, to ensure a desired level of data security.

Exemplary Modules of the Present Invention

A patient meets with a mental health professional in-person for one or more clinical and/or diagnostic tests. The patient is provided with a personal digital assistant, such as a smartphone, with wireless connectivity to a communication network, such as the Internet. The mental health professional or an agent thereof (such as a clinical assistant) is located at a location remote from the patient, having access to a computer connected to the internet.

The patient is prescribed an exposure therapy module using the present invention, which involves determining and treating avoidance tactics employed by the patient. For example, the patient and the mental health professional can interact remotely through the Internet, such that the mental health professional can determine the etiology of the psychological condition, e.g., the traumatic stimulus or event experienced by the patient, and prompt the patient to engage in therapeutic thoughts and/or condition with respect to the stimulus or event. In preferred embodiments, the mental health professional engages the patient in activities, experiments, exercises, and/or discussions to treat the patient's thoughts, and corresponding actions, related to the stimulus or event, preferably to make sense of the stimulus or event. In some embodiments, the mental health professional engages with a patient to develop an exposure hierarchy, in which degrees of the traumatic stimulus or event are ranked from easiest to handle to hardest to handle.

In certain embodiments, the mental health professional teaches coping skills, such as techniques for managing stress (i.e., breathing techniques) and then exposes the patient to one item from the hierarchy at a time, starting with the easiest. For example, the mental health professional helps the patient to relax while the feared stimulus is presented and keeps the patient in the presence of the stimulus until the fear level goes down. In yet additional embodiments, the mental health professional leads the patient through prolonged exposure that guides the patient through thought of the stimulus or event repeatedly.

Specific treatment techniques known to one skilled in the art may be adapted for use in accordance with embodiments of the present invention. For example, Foa, E. B., Hembree, E. A., & Rothbaum, B. O., “Prolonged Exposure Therapy for PTSD: Emotional Processing of Traumatic Experiences Therapist Guide,” Oxford University Press, New York, 2007, is herein incorporated by reference in its entirety.

### Example 1

A wireless, cellular based-device would be provided to a patient in need, such as an active, reserve, or retired member of the military (i.e., soldier) with PTSD or acute stress disorder. The device would include a GPS locator, which may be used to assist emergency services and keep the device from being lost or misplaced. If the device is lost or compromised, the device would include a kill (or suicide) switch which may be remotely activated. Additionally, the device would include a speed or direct dial to 911 or emergency services.

The device would be pre-loaded with a complete library of assessment, screening, monitoring and diagnostic instruments associated with PTSD and other traumatic stress injuries. The device may further include preloaded homework and practice assignment for anxiety and affective disorders including PTSD, depressive disorders and anxiety family disorders. The device may also provide deep-breathing and relaxation exercises, progressive muscle relaxation, guided imagery and biofeedback mechanisms for individuals with military traumatic stress injuries.

### Example 2

The patient device would include several modules. The patient device would include a psychoeducation module, in which patients would be able to access scientifically and psychologically accurate information to inform them and their families about symptoms they may be experiencing. The device would be further equipped with treatment options: patients will be able to utilize applications to access treatment strategies, including biofeedback, progressive muscle relaxation, deep breathing, imaginal exposure, guided imagery. A skills-based module would allow patients to practice skills learned in therapy and complete various homework (preferably interactive) assignments (Cognitive Process writing, ABC worksheets or Behavioral Activation Exercises). Patients would also be able to complete evidence based instruments from a library of options and prescribed by clinicians (PCL, BDI, BAI, BSS, ASI, etc.)

Clinicians, using one or more networked personal computers, would be able to communicate with the patient device at anytime, 24-hours a day, seven days a week. Data would be temporarily stored on the patient device and transferred to a secure database at a predetermined time or at the option of the clinician or patient. The database would be securely accessible by the clinicians, who would monitor and track patient progress by having patients complete various screening instruments from a comprehensive library (PCL, BDI, BAI, BSS, BHS, ASI, etc.), as would be prescribed by a clinician. Data from assessments could be transferred to a central repository that clinicians will be able to access and utilize to mark treatment development. Clinicians could use the data to conduct micro and macro analysis of patient status. In accordance with HIPPA, data on this device would only be accessed by appropriate and identified healthcare providers through use of a secure database. Patients would be informed of their rights under the HIPPA statute as part of the consenting procedure.

Exemplary Computer Implementation Embodiment

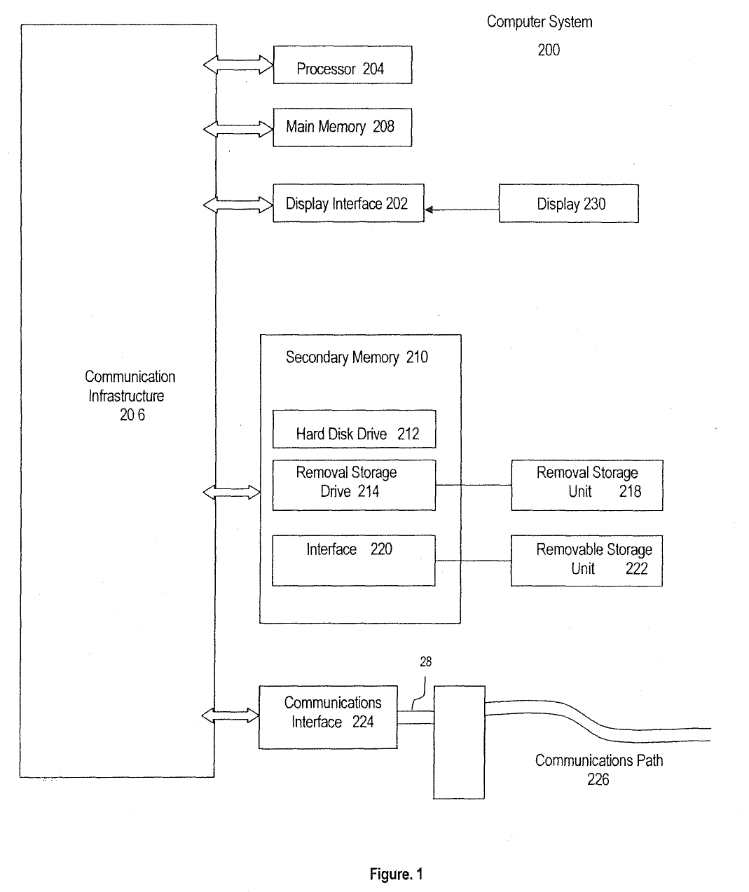

As indicated above, aspects of methods or systems of the present invention may for example be electronically implemented using hardware, software, or a combination thereof and may be implemented in one or more computer systems or other processing systems. In one embodiment, computer systems capable of carrying out the functionality described herein. An example of such a computer system is shown in FIG. 1.

Computer system 100 includes a central processing unit (CPU) one or more processors, such as processor 104 for controlling the operation of the system. Processor 104 is connected to a communication infrastructure 106 (e.g., a communications bus, cross-over bar, or network). Various software embodiments are described in terms of this exemplary computer system. After reading this description, it will become apparent to a person skilled in the relevant art(s) how to implement systems or methods of embodiments of the present invention using other computer systems and/or architectures.

Computer system 100 can include a display interface 102 that forwards graphics, text, and other data from the communication infrastructure 106 (or from a frame buffer) for display on the display unit 130. Display interface 102 can include or involve processor 104, main memory 108, or other components in providing such functionality. Computer system 100 also includes a main memory 208, preferably random access memory (RAM), and may also include a secondary memory 110. The secondary memory 110 may include, for example, a hard disk drive 112 and/or a removable storage drive 114, representing a floppy disk drive, a magnetic tape drive, an optical disk drive, etc. The removable storage drive 214 reads from and/or writes to a removable storage unit 118 in a well-known manner. Removable storage unit 118, represents a floppy disk, magnetic tape, optical disk, etc., which is read by and written to removable storage drive 114. As will be appreciated, the removable storage unit 118 includes a computer usable storage medium having stored therein computer software and/or data.

In alternative embodiments, secondary memory 110 may include other similar devices for allowing computer programs or other instructions to be loaded into computer system 100. Such devices may include, for example, a removable storage unit 122 and an interface 120. Examples of such may include a program cartridge and cartridge interface (such as that found in video game devices), a removable memory chip (such as an erasable programmable read only memory (EPROM), or programmable read only memory (PROM)) and associated socket, and other removable storage units 122 and interfaces 120, which allow software and data to be transferred from the removable storage unit 122 to computer system 100.

If desired, some or all pertinent software, processing, and/or data can be performed or implemented by a hosting service (e.g., application or data host) such as a provider that has an array of servers connected to the Internet. Such implementations are sometimes referred to as cloud computing 111.

Computer system 200 may also include a communications interface 124. Communications interface 124 allows software and data to be transferred between computer system 100 and external devices. Examples of communications interface 124 may include a modem, a network interface (e.g., an Ethernet card), a communications port, a Personal Computer Memory Card International Association (PCMCIA) slot and card, etc. Software and data transferred via communications interface 124 are in the form of signals 128, which may be electronic, electromagnetic, optical or other signals capable of being received by communications interface 124. These signals 128 are provided to communications interface 124 via a communications path (e.g., channel) 126. This path 126 carries signals 128 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 114, a hard disk installed in hard disk drive 112, and signals 128. These computer program products provide software to the computer system 100.

Computer programs (also referred to as computer control logic) are stored in main memory 108 and/or secondary memory 110. Computer programs may also be received via communications interface 124. Such computer programs, when executed, enable the computer system 100 to perform the features of embodiments of the present invention, as discussed herein. In particular, the computer programs, when executed, enable the processor 104 to perform the features of embodiments of the present invention. Accordingly, such computer programs represent controllers of the computer system 100.

In an embodiment where the invention is implemented using software, the software may be stored in a computer program product and loaded into computer system 100 using removable storage drive 114, hard drive 112, or communications interface 124. The control logic (software), when executed by the processor 104, causes the processor 104 to perform the functions of the invention as described herein. In another embodiment, the invention is implemented primarily in hardware using, for example, hardware components, such as application specific integrated circuits (ASICs). Implementation of the hardware state machine so as to perform the functions described herein will be apparent to persons skilled in the relevant art(s). In practice, the system will typically take the form of a personal computer running Windows, but may take the form of a Unix workstation, a mainframe computer, a mid-range computer, or any other apparatus that can is be configured to perform the functions described herein. The software described herein may be written in any programming language that is compatible with the system.

In yet another embodiment, a combination of both hardware and software may be used for implementing such features.

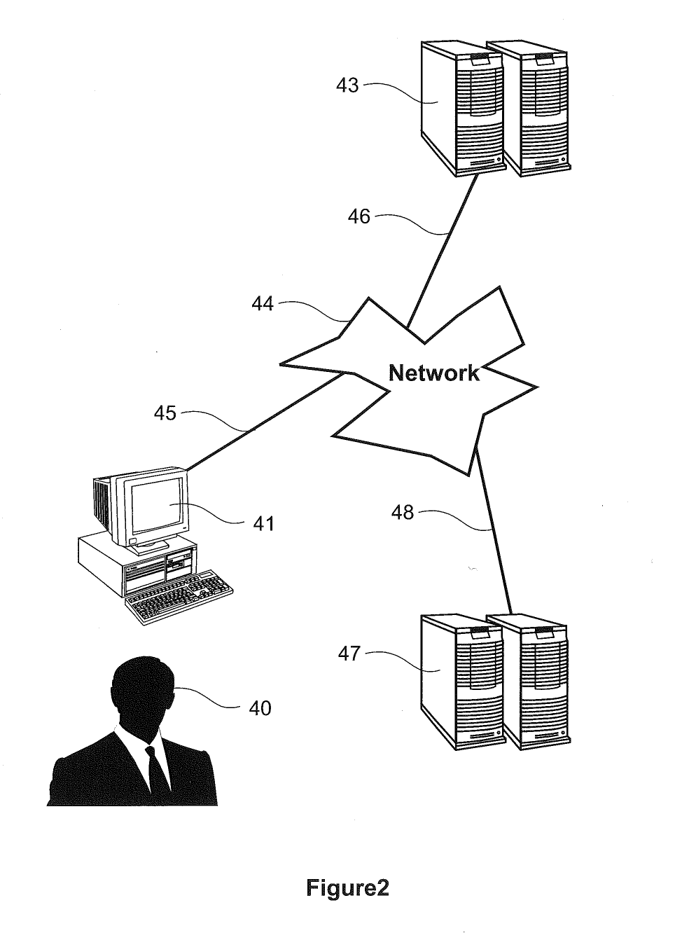

As shown in FIG. 2, in an embodiment of the present invention, the system and process of the present invention operates, for example, on a network. A user 40, such as an patient or patient processor inputs information, such as through a keyboard, mouse, touchscreen, or the like, via a terminal 41, such as a personal computer (PC), minicomputer, mainframe computer, microcomputer, telephone device, personal digital assistant (PDA), multi-use gaming device, or other device having a processor and input capability.

As further shown in FIG. 2, in one embodiment, the terminal 41 is coupled to a server 43, such as a PC, minicomputer, mainframe computer, microcomputer, or other device having a processor and a repository for data or connection to a repository for maintained data, via a network 44, such as the Internet, via couplings 45, 46, such as wired, wireless, or fiber optic connections.

Thus, in accordance with the foregoing the objects of the present invention are achieved. Modifications to the above would be obvious to those of ordinary skill in the art, but would not
bring the invention so modified beyond the scope of the appended claims.

What is claimed is:

1. A computer-implemented system for monitoring post-traumatic stress disorder in a patient in need, comprising:
an electronic, portable patient device having at least an input mechanism to enter patient information, a display to review the patient information and communication apparatus; and
a computer-implemented processor accessible by one or more mental health professionals and located remote from the patient device, the computer-implemented processor configured to transmit one or more executable programs in a patient clinical module to the patient device to receive executed programs from the patient,
wherein the patient clinical modules provide therapies for mental health care for post-traumatic stress disorder or symptoms thereof, selected from the group comprising: a therapy module to monitor and detect a post-traumatic stress disorder symptoms; an informational module to deliver educational material about post-traumatic stress disorder; and
wherein the system distributes one or more of these modules to the patient device and can connect patients to health care providers.

2. The system of claim 1, wherein the patient is an adult associated with the military.

3. The system of claim 1, wherein the patient's post-traumatic stress disorder corresponds to a military-related exposure.

4. The system of claim 1, wherein the patient clinical module is transmitted from the computer-implemented processor to the patient device through the communication apparatus.

5. The system of claim 4, wherein contents of the patient clinical module is customized to each patient device.

6. The system of claim 1, wherein the patient clinical module is installed through a removable memory.

7. The system of claim 1, wherein herein the communication means is wireless.

8. The system of claim 1, wherein the communication apparatus of patient device comprises an alert function to alert the healthcare worker of symptoms exceeding predetermined threshold levels corresponding to post-traumatic stress disorder.

9. The system of claim 1, wherein the computer-implemented processor communicates with one or more military databases with patient information.

10. The system of claim 1, the computer-implemented processor communicates with a third-party associated with patient mental healthcare through a communication network, wherein the computer-implemented processor comprises an alert function to electronically alert a first responder party through the communication network when the patient exceeds at least one predetermined threshold diagnostic measurement level corresponding to post-traumatic stress disorder or is deemed by the mental health professional to be having a mental health emergency.

11. The system of claim 1, wherein the handheld device further comprises a security mechanism, wherein patient information cannot be accessed upon activation of the security mechanism.

12. The system of claim 1, wherein a monitoring program comprises:
a self-report module, wherein a patient inputs information relating to the patient's emotional and mental health status;
a skills building program having at least one breathing techniques program; and
a cognitive behavioral therapy, wherein the cognitive behavioral therapy includes an inquiry regarding an exposure related to one or more symptoms of post-traumatic stress disorder.
wherein modules made available as patient's health status changes.

13. The system of claim 1, wherein the patient device displays each program of the patient clinical module as a component corresponding to the patient's o mental health therapy.

14. The system of claim 1, wherein the computer-implemented device is connectable to at least 2 patient devices simultaneously.

15. The system of claim 1, wherein the computer-implemented device is connectable to at least 5 patient devices simultaneously.

16. The system of claim 1, wherein the computer-implemented device is connectable to at least 10 patient devices simultaneously.

17. A method for treating post-traumatic stress disorder in a patient in need, comprising:
providing a patient device to the patient to collect patient information regarding post-traumatic stress disorder;
providing a post-traumatic stress disorder screening functionality on the handheld device, wherein the screening functionality is configured with threshold clinical and diagnostic values to determine a degree of post-traumatic disorder symptoms;
receiving collected patient information through a communication network on at least one computer-implemented processor accessible by a mental health professional;
processing the patient information to determine the clinical degree of post-traumatic stress disorder; and
optionally alerting a first responder if one or more predetermined threshold clinical values are exceeded by the patient.

18. The method of facilitating regimen for remotely monitoring post-traumatic stress disorder in a patient, comprising:
providing an entity associated with at least one mental health professional with a system to remotely diagnose and monitor post-traumatic stress syndrome in at least one patient through one ore more computer-executable programs directed to post-traumatic stress disorder, the programs prompting patient interaction from the patient through a communication network connecting a patient device with a central monitoring computer, wherein the patient device and central monitoring computer transmit and receive patient mental health assessment information providing information on the patient's mental health
collecting the information at the central monitoring computer as a component of a patient profile for treatment of post-traumatic stress disorder;
assessing the information to determine efficacy and scope of the treatment; and
customizing the treatment by updating one or more computer-executable programs to be addressed by the patient as part of therapy for post-traumatic stress disorder.

19. A post-traumatic stress disorder treatment and monitoring device, comprising:
a patient-controlled electronic device configured to provide one or more patient-directed modules related to post-traumatic stress disorder, the patient-controlled device having at least an input mechanism to input data, a display to view data and a communication apparatus to permit communication through a communication network; and
a screening module configured to identify post-traumatic stress disorder or symptoms thereof and configured to perform an output operation in response to detecting post-traumatic stress disorder or at least one symptom thereof;
a therapy module configured to provide treatment for post-traumatic stress disorder and configured to perform an output operation in response to patient response to therapy;
a communication module configured to receive and transmit patient input and configured to perform an output operation in response to accepting a patient input; and
a skills-building module configured to train patients on coping with post-traumatic stress disorder and configured to perform an output operation in response to patient input.

20. An article of manufacture comprising:
a computer readable medium; and
a data structure stored thereon adapted and configured to route signals, wherein the data structure comprises a computer readable system for treating and monitoring post-traumatic stress disorder in patients in need:
a processing system configured and adapted to communicate with a plurality of computers, wherein the processing system is arranged to accept input of:
one or more an electronic, portable patient devices having at least an input mechanism to input data, a display to view data and communication apparatus; and
a computer-implemented processor accessible by one or more mental health professionals and located remote from the patient device, the computer-implemented processor configured to transmit one or more executable programs in a patient clinical module to the patient device to receive executed programs from the patient,
wherein the patient clinical modules provide therapies for mental health care for post-traumatic stress disorder or symptoms thereof, selected from the group comprising: a therapy module to monitor and detect a post-traumatic stress disorder symptoms; an informational module to deliver educational material about post-traumatic stress disorder.
